(19) United States
(12) Patent Application Publication
(10) Pub. No.: US 2010/0175699 A1
(43) Pub. Date: Jul. 15, 2010

(54) RESPIRATORY SENSOR

(75) Inventors: Mark Varney, Hampshire (GB); Michael Garrett, Surrey (GB); Deryk Williams, Surrey (GB); John Beadle, Essex (GB)

Correspondence Address: Kimberly A. Chasteen, PO Box 1243, Yorktown, VA, 23692, US

(73) Assignee: ANAXSYS TECHNOLOGY LTD, Send, Nr Working, Surrey (GB)

(21) Appl. No.: 12/595,536

(22) PCT Filed: Apr. 10, 2008
(86) PCT No.: PCT/GB2008/001251

§ 371 Date: Mar. 22, 2010

(30) Foreign Application Priority Data
Apr. 10, 2007 (GB) 0706881.0
Aug. 8, 2007 (GB) 0715584.9

(51) Int. Cl. A61M 16/00; A61B 5/08; A61B 5/02

(52) U.S. Cl. 128/204.23; 600/529; 600/484

(57) ABSTRACT

A sensor assembly for monitoring the respiration pattern of a subject is disclosed, the sensor comprising a sensor element for exposing to a gas stream exhaled by the subject comprising a first electrode, a second electrode and an active layer extending between the first and second electrodes to provide an electrical path therebetween, the sensor element being responsive to the concentration of water vapour in the gas stream exhaled by the subject, whereby the conductivity of the path between the electrodes varies in response to changes in the concentration of water vapour; a processor for determining the pattern of respiration of the subject; and means for displaying information relating to the determined respiration pattern to a user. The sensor assembly is particular suitable for measuring the respiration rate and/or depth of respiration of the subject. The sensor and method find particular application in the diagnosis and treatment of sleep apnea.

The present invention relates to a sensor and method for monitoring and measuring the breathing patterns of a subject, including the depth of respiration and the respiratory rate of a subject. The present invention finds use in the monitoring of the respiration of a subject in general and in particular to the monitoring and identification of interruptions in the breathing pattern of the subject, for example as a result of sleep apnea and other like conditions.

The pattern of respiration exhibited by a subject can be affected by a range of significant conditions affecting the health of the subject and an understanding of such respiration patterns can be a very useful guide in the diagnosis and treatment of many conditions. Respiratory rate is one of the most important physiological parameters reflecting the spontaneous living status of a subject. It is a vital component of most medical and nursing records, and is used in many clinical scoring systems. Extremes of respiratory rate usually indicate the need for urgent medical intervention. Typically, the measurement of respiratory rate is based on human observation and this is the sole technique used for determining respiratory rate in very many cases.

Sleep apnea is an episodic upper airway obstruction occurring during sleep, and afflicts an estimated 1% to 5% of the general population. Those afflicted with sleep apnea experience sleep fragmentation and intermittent, complete or nearly complete cessation of ventilation during sleep with potentially severe degrees of oxyhemoglobin desaturation. These features may be translated clinically into extreme daytime sleepiness, cardiac arrhythmias, pulmonary-artery hypertension, congestive heart failure and/or cognitive dysfunction. Other sequelae of sleep apnea include right ventricular dysfunction with cor pulmonale, carbon dioxide retention during wakefulness as well as during sleep, and continuous reduced arterial oxygen tension. Hypersomnolent sleep apnea patients may be at risk for excessive mortality from these factors as well as by an elevated risk of accidents while driving and/or operating potentially dangerous equipment.

Although details of the pathogenesis of upper airway obstruction in sleep apnea patients have not been fully defined, it is generally accepted that the mechanism includes either anatomic or functional abnormalities of the upper airway which result in increased air flow resistance. Such abnormalities may include narrowing of the upper airway due to suction forces evolved during inspiration, the effect of gravity pulling the tongue back to appose the pharyngeal wall, and/or insufficient muscle tone in the upper airway dilator muscles. It has also been hypothesized that a mechanism responsible for the known association between obesity and sleep apnea is excessive soft tissue in the anterior and lateral neck which applies sufficient pressure on internal structures to narrow the airway.

The treatment of sleep apnea has included such surgical interventions as uvulopalatopharyngoplasty, gastric surgery for obesity, and maxillo-facial reconstruction. Another mode of surgical intervention used in the treatment of sleep apnea is tracheostomy. These treatments constitute major undertakings with considerable risk of postoperative morbidity if not mortality. Pharmacologic therapy has in general been disappointing, especially in patients with more than mild sleep apnea. In addition, side effects from the pharmacologic agents that have been used are frequent.

Thus, the medical fraternity continues to seek non-invasive modes of treatment for sleep apnea with high success rates and high patient compliance including, for example in cases relating to obesity, weight loss through a regimen of exercise and regulated diet.

It is known in the art to monitor patients susceptible to disorders of the respiratory system or in critical care environments and to employ respiratory sensors and alarm systems. In general, a change in an individual's respiration (known as “apnea” when such a change is a transient cessation of respiration) corresponds to a change in the physical condition of that individual. In the case of individuals with obstructive sleep apnea, a change in respiration may be attributable to present or impending physical distress. Accordingly, there is a need for a simple and reliable method of sensing respiration, and changes in respiration, that will meet medical and clinical needs for monitoring individuals at risk of respiratory distress. There is an especial need to monitor new-born infants.

There have been a multitude of airflow sensors employed to measure the volume and rate of breathing. These are variously based on physical measurements (light, sound, pressure, air velocity, etc.) and chemical measurements (gas sensing, temperature, infrared light absorption, etc.).

For example, EP 0484174 describes a complex assembly for monitoring the respiration of a patient in the form of a face mask. The assembly comprises a thermistor which, in use, is exposed to the inhaled and exhaled gas streams of the patient. The assembly includes both a visual and audible indication of respiration, with an alarm sounding in the case that the patient stops breathing.

Further, U.S. Pat. No. 5,311,875 and U.S. Pat. No. 7,089,932 describe the use of polyvinylidene fluoride (PVDF) as suitable transducer films for sensing the temperature difference between inspired and expired breaths.

Other possible solutions to the problem of monitoring the respiration of a subject include spectroscopic (infrared) end-tidal CO₂ monitors, catheterization, and (simple) visual observation. Such measurements are either ‘invasive’ (and cause e.g. back-pressure and therefore change the subjects' breathing), and/or are expensive/complicated to implement into a handheld, battery-operated instrument, and/or are subject to component failure for one reason or another.

Thus there is a need in the field of medical healthcare for a simple, inexpensive, robust sensor for the non-invasive measurement of breathing pattern of a subject, including depth of breathing and respiratory rate, and which does not require artificial sources of radiation or complex equipment. It would be advantageous if the sensor could be self-contained, in particular be able to be powered by a battery. Further, the sensor should not be invasive so as to affect or interfere with the breathing pattern of the subject.

Accordingly, in a first aspect, the present invention provides a sensor assembly for monitoring the respiration pattern of a subject, the sensor comprising:

a sensor element for exposing to a gas stream exhaled by the subject comprising a first electrode, a second electrode and an active layer extending between the first and second electrodes to provide an electrical path therebetween, the sensor element being responsive to the concentration of water vapour in the gas stream exhaled by the subject, whereby the conductivity of the path between the electrodes varies in response to changes in the concentration of water vapour;

a processor for determining the pattern of respiration of the subject; and

means for displaying information relating to the determined respiration pattern to a user.

The sensor of the present invention allows the breathing or respiration pattern of a subject to be monitored by an analysis of the gas stream exhaled by the subject. In particular, the sensor may be used to measure the rate of respiration of the subject over a plurality of successive inhalations and exhalations. The sensor may also be used to measure the depth of respiration of the subject, which may be carried out by analysis of a single exhalation or a plurality of exhalations of the subject.

The present invention overcomes the disadvantages found in the prior art by providing a measurement of breathing patterns, in particular a respiratory rate measurement, derived from the measurement of exhaled water vapour, by means of an electrochemical sensor element mounted so as to be exposed to the exhaled gas stream of a living subject. Moreover, the present invention includes a sensor construction which is less susceptible to interference from the external environment, motion of the subject, and is, as a result, less prone to a loss of signal and output to the user.

As used herein that the terms “respiration rate” and “respiratory rate” are synonymous. In addition, as used herein the term “respiratory rate” is defined as the number of breaths taken by the subject per minute (bpm).

The sensor element of the assembly of the present invention is particularly suitable for the detection of water vapour present in the exhaled breath of a person, or animal. In addition, the sensor provides a fast and accurate response to changes in the water vapour content of the gas stream being analysed. These features make the sensor assembly of the present invention particularly suitable for use as a sensor for respiratory rate and other breathing patterns in the analysis of exhaled breath of a subject.

The present invention provides a sensor assembly that may be particularly compact and of very simple construction. In addition, the sensor assembly may be used at ambient temperature conditions, without the need for any heating or cooling, while at the same time producing an accurate measurement of water vapour concentration in the gas being analysed.

Breathing by the subject through the sensor assembly may be wholly nasal, wholly oral, or a combination of both. The sensor assembly is preferably arranged to allow the user to switch between the various modes or breathing, either wholly nasal, mixed nasal and oral, and wholly oral, particularly during sleep.

A problem for workers in the field has been ensuring that the breath of the subject always impinges upon the sensors of known devices. Accordingly, to avoid any false readings, the sensor element may be incorporated within a breathing tube, a cannula, mask or other apparatus formed on or around the face. Preferably, the sensor element is mounted within a standard 20 mm id tubing adaptor, such that it can be easily mounted within a mask or fitted into commercially available breathing lines and tubing.

Whilst the use of a face mask channels respiratory flow to the sensor, a face mask may be unsuitable for some human subjects and/or animals. Accordingly, in one embodiment, the sensor element of the assembly of the present invention is arranged to be mountable to the subject, so as to cause a gas stream exhaled through the nose of the subject to impinge upon the sensor element. This embodiment of the invention may be preferred by some users, as it provides a less intrusive solution. For example, the sensor assembly may be adapted so that the sensor element can be placed adjacent to the nostrils, for example taped to the upper lip and oriented so that the sensor is directly underneath a nostril.

In one particularly preferred arrangement, the sensor element is housed within an open ended conduit disposed on the upper lip of the subject and extending longitudinally between the nose and mouth of the subject. It has been found that such an arrangement allows a portion of the gas streams being inhaled and exhaled to be caused to flow through the conduit and contact the sensor element, regardless of whether the subject breathes through their mouth or their nose. In this way, the subject is not impeded in their choice of breathing manners.

The sensor assembly comprises a processor and means for displaying information relating to the respiration of the subject, in particular the respiration rate. The sensor element, processor and display means may be interconnected by any suitable means and suitable arrangements are known in the art. In one embodiment, the sensor element is wirelessly connected to a suitable signal recording, processing and display system.

The sensor element of the sensor assembly may be independently mounted and used with the subject, either alone or in addition to other devices and instruments, such as lines for supplying gases, such as oxygen, to the subject, as frequently may be required. Alternatively, in one arrangement, the sensor element of the assembly may also be mounted within standard gas delivery lines such as oxygen.

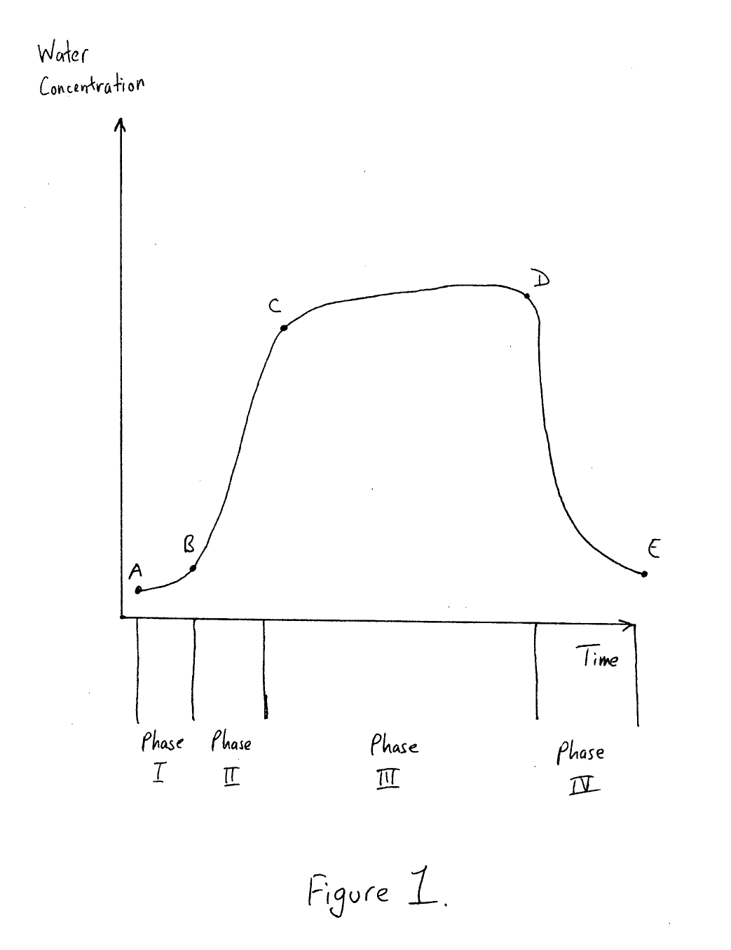

The sensor assembly monitors the respiration of the subject, in particular the extent and/or rate of breathing, by measuring the changes in humidity, as a function of tidal volume and/or time. In the case of tidal breathing, the sensor element of the assembly is at least exposed to the gas streams exhaled by the subject. In this case, the sensor element will respond to the changes in humidity or water vapour concentration that occur over the time span of an exhaled breath. In particular, the concentration of water vapour in an exhaled breath when plotted against time follows a generally rectangular trace. A typical trace of the concentration of water vapour plotted against time for a single exhaled breath is shown in FIG. 1. As can be seen, the trace comprises an ascending phase, with the concentration increasing rapidly with time as subject breathes out. There follows a plateau phase, with the concentration changing significantly less than the ascending phase. Finally, towards the end of the breath, the concentration falls rapidly in a descending phase. The ascending phase and plateau phase are particularly affected by changes in ventilation (V) and perfusion (Q) of the subject. Accordingly, the sensor assembly of the present invention may determine the rate of respiration of the subject by identifying one or more significant features in the signal data generated by the sensor element and as represented graphically in FIG. 1, for example one or more of the end of the ascending phase, the start of the descending phase, the peak concentration achieved during the plateau phase, and the like. In this manner, successive breaths of the subject can be readily identified and a determination of the rate of respiration made.

Further, the data generated by the sensor assembly of the present invention may be used to determine the depth of breathing, that is the volume of gas being inhaled and exhaled by the subject. In particular, the data relating to change of concentration of water vapour over time can be integrated to determine the volume of gas being exhaled by the subject, as represented by the area under the curve of FIG. 1, for example. The depth of breathing can provide an indication of certain conditions within the respiratory system of the subject and changes in the depth of breathing can be monitored and used to identify the onset or change in a condition. For example, in the case of a subject suffering from sleep apnea, it is often the case that an apneic event is preceded by an erratic change in the volume of gas being expired by the subject, that is the subject's breathing becomes shallower. The sensor assembly of the present invention allows for such events to be identified and the imminent onset of an apneic event determined, in turn allowing for some corrective action to be taken.

More preferably, the sensor element of the sensor assembly of the present invention is exposed to gas streams that are both inhaled and exhaled by the subject. The concentration of water vapour in the inhaled gas stream will be that of the ambient atmosphere surrounding the subject and will have a substantially constant concentration of water vapour, at least in comparison with the varying concentration of water vapour in the exhaled gas streams.

From a consideration of the profile of changes in the concentration of water vapour in the gas streams being exhaled and, if appropriate, inhaled by the subject, it follows that the sensor element needs to respond to a wide range of humidity conditions during the breathing of the subject. In this respect, it has been found advantageous to provide means for at least partially drying the sensor element between the exhaled breaths of the subject. The sensor element is preferably dried to remove as much water vapour from the element itself and the surrounding atmosphere as possible, at least to reduce the concentration of water vapour to below the minimum concentration that will be encountered by the sensor element being exposed to the breath of the subject. Any suitable means for drying the sensor element may be employed. The drying means may be operable to dry the sensor after each exhalation of the subject, for example during a part or the whole of the succeeding inhalation. Alternatively, the drying means may be operable periodically, for example after a specified number of exhalations, for example after each 5, more preferably each 3 exhalations.

The drying of the sensor element may be triggered by any suitable means. One preferred arrangement is to have the drying means triggered to begin drying by the detection of the flow reversal of the gas stream. In this way, the drying means remains dormant while the subject is exhaling. Upon inhalation, the direction of flow of the gas through the sensor assembly will be reversed. Detection of flow reversal can be used to start the drying sequence for the sensor element.

In one embodiment, the sensor element is disposed so as to be contacted by the gas stream inhaled by the subject. In this way, the relatively low humidity gas stream being inhaled is used to remove moisture from the sensor element.

Alternatively, the sensor assembly may be provided with a supply of pressurized gas, a stream of which is directed to impinge upon the sensor element. The flow of pressurized gas may be intermittent with the pressurized gas being supplied only during the periods in which the subject is exhaling. More preferably, the flow of pressurized gas is continuous, thereby providing a constant feed of dry gas to the sensor element. In such a case, the volumetric flowrate of the pressurized gas is sufficiently high to remove water from the sensor element to the required level, while being sufficiently low to not interfere with the response of the sensor element to the changes in concentration of water vapour in the exhaled gas stream of the subject. The pressurized gas may be any suitable gas, for example air. One preferred gas is oxygen or oxygen-enriched air, which is frequently supplied to a subject to assist with many conditions. In such a case, the sensor assembly may be arranged such that the pressurized gas may be inhaled by the subject. In this arrangement, it is preferred that the supply of pressurized gas is divided into two streams, a first major stream being supplied directly for inhalation by the subject, and a second minor stream directed to the sensor element to perform the drying function. The ratio of flowrates of the first and second streams may be in the range of from 5:1 to 25:1, more preferably from 10:1 to 20:1.

The sensor assembly comprises a sensor element having a plurality of electrodes with an active layer extending therebetween. The active layer comprises a material that reacts to the presence of water vapour in the gas stream contacting the sensing element, so as to change the conductivity of the electrical path between the electrodes. The active layer, by being selective in its activity to water vapour, provides the sensor element with the ability to measure the quantity of water vapour in the gas stream being analysed or monitored. It follows that two or more similar sensor elements may be combined, each having a coating with a different activity, in order to increase the confidence of water vapour detection.

The conductivity of the electrical path between the electrodes changes as a result of changes in the concentration of water vapour, which in turn affect the impedance of the electrical path. The changes in impedance may be changes in one or more of resistance, capacitance and inductance. In a preferred embodiment, the layer of material extending between the electrodes is active with respect to water vapour, such that the conductance of the electrical path established between the electrodes is proportional to the concentration of water vapour in the gas stream.

Suitable components for inclusion in the active material include Zeolites and silicalites. Suitable Zeolites include the naturally occurring and synthetic Zeolites. The methods of preparing suitable Zeolites are well known in the art and suitable Zeolites for use in the sensor of the present invention are available commercially.

The active material is preferably porous, with Zeolites being one particularly preferred porous material. Zeolites, being highly porous materials belonging to the class of aluminosilicates have been found to be particularly suitable for use as or comprised in the active material of the sensor element of the present invention. Zeolites are characterized by having a crystalline structure with a 3-dimensional pore system. The pores are precisely defined in terms of their diameter. The diameter of the pores may be controlled by subjecting the Zeolite to ion-exchange with appropriate cations, using techniques well known in the art. It has been found that the speed of response of the sensor element is, in part, dependant upon the relationship of the pore size of the active material with the diameter of the target species or molecule in the gas stream being analysed. In particular, it has been found that active materials, especially Zeolites, having pores with a diameter significantly greater than that of the target molecule give rise to a sluggish or slow response of the sensor element to changes in the composition of the gas stream being analysed. In contrast, the speed of response of the sensor element increases as the pore diameter approaches the diameter of the target molecule. Accordingly, it is preferred that the diameter of the pores of the active material are not substantially greater than the diameter of the target species or molecule. More preferably, the nature of the active material can be chosen by experiment, choosing that which has the fastest speed of response.

Suitable Zeolites for use in the sensor element of the present invention include the Type A, Type P and Type X Zeolites. Preferred Zeolites include Zeolite 4A and 13X. Zeolite 4A, having a pore diameter of about 4 Angstroms, is a particularly preferred Zeolite for use when the target molecule in the gas stream is water vapour.

Suitable Zeolites for use in the sensor element are known in the art and available commercially, or may be prepared using techniques and methods well known in the art.

Synthetic aluminium-magnesium hydroxycarbonates, in particular hydrotalcite, are also suitable for use in the active material.

In one preferred embodiment of the present invention, the electrodes are in contact with, for example coated with, a layer of ion exchange material extending between the working electrode and the counter electrode; whereby contact of the ion exchange layer with the gas stream forms an electrical contact between the working and counter electrodes.

In the present specification, references to an ion exchange material are to a material having ion exchange properties, such that contact with the components of a gas stream results in a change in the conductivity of the layer between the electrodes. The ion exchange material acts as the support medium for electrical conduction to occur, as it allows a hydrated ionic layer to form between the electrodes. The layer of ion exchange material provides a medium that is highly controllable and hydrates uniformly to provide a suitable medium for conduction to occur.

Suitable ion exchange materials for use in the sensor of the present invention are those having a high proton conductivity, good chemical stability, and the ability to retain sufficient mechanical integrity. The ion exchange material should have a high affinity for the species present in the gas stream being analysed, in particular for the various components that are present in the exhaled breath of a subject or patient.

Suitable ion exchange materials are known in the art and are commercially available products.

Particularly preferred ion exchange material are the ionomers, a class of synthetic polymers with ionic properties. A particularly preferred group of ionomers are the sulphonated tetrafluoroethylene copolymers. An especially preferred ionomer from this class is Nafion®, available commercially from Du Pont. The sulphonated tetrafluroethylene copolymers have superior conductive properties due to their proton conducting capabilities. The sulphonated tetrafluroethylene copolymers can be manufactured with various cationic conductivities. They also exhibit excellent thermal and mechanical stability and are biocompatible, thus making them suitable materials for use in the controlled electrode coating.

Other suitable ion exchange materials include polyether ether ketones (PEEK), poly(arylene-ether-sulfones) (PSU), PVDF-graft styrenes, acid doped polybenimidazoles (PBI) and polyphosphazenes.

The ion exchange material may be present in the sensor in the dry state. Alternatively, the ion exchange material may be present with water in a saturated or partially-saturated state.

The thickness of the ion exchange material will determine the response of the sensor element to changes in the composition of the gas stream in contact with the ion exchange layer. To minimize internal resistance within the sensor element, it is preferred to use an ultra thin ion exchange layer.

The ion exchange layer may comprise a single ion exchange material or a mixture of two or more such materials, depending upon the particular application of the sensor.

The ion exchange layer may consist of the ion exchange material in the case the material exhibits the required level of chemical and mechanical stability and integrity for the working life of the sensor. Alternatively, the ion exchange layer may comprise an inert support for the ion exchange material. Suitable supports include oxides, in particular metal oxides, including aluminium oxide, titanium oxide, zirconium oxides and mixtures thereof. Other suitable supports include oxides of silicon and the various natural and synthetic clays.

In one preferred arrangement, the electrodes of the sensor element are coated with a layer consisting essentially of one or more ion exchange materials. Ion exchange materials, such as the commercially available sulphonated tetrafluoroethylene copolymers, in particular the Nafion® product, can be deposited onto the sensor element to coat and cover the electrodes providing a tough layer that is resistant to abrasion, cracking and sloughing. The sensor assembly will require repeated inhalations and exhalations by the subject in order to fully determine the respiratory rate. The sensor element of this arrangement provides a particularly strong and hardy device capable of withstand repeated usage over an extended period of time.

In a further preferred embodiment, the electrodes of the sensor element are in contact with, for example coated in, a layer of mesoporous material extending between the working electrode and the counter electrode; whereby contact of the mesoporous layer with the gas stream forms an electrical contact between the working and counter electrodes.

In the present specification, references to a mesoporous material are to a material having pores in the range of from 1 to 75 nm, more particularly in the range of from 2 to 50 nm. The mesoporous material acts as the support medium for electrical conduction to occur, as it allows a temporary hydrated ionic layer to form across the electrodes. The layer of mesoporous material provides a medium that is highly controllable and hydrates uniformly to provide a suitable medium for conduction to occur.

Suitable mesporous materials for use in the sensor of the present invention include metal oxides, in particular oxides of metals from Group IV of the Periodic Table of the Elements, in particular oxides of titanium or zirconium. A particularly preferred metal oxide is titanium oxide, including the titanates. Alternative mesoporous materials of use are synthetic clays, of particular preference due to the inherent layered nature of the clays. Laponite is a synthetic layered silicate with a structure resembling that of the natural clay mineral, hectonite. When added to water with stirring it will disperse rapidly into nanoparticles. It is cost effective, heat stable, thixotropic and can retain levels of hydration. Laponite is of special interest because of its single ion conducting character, where concentration polarization can be minimised. Hydrotalcite-like compounds are known also as layered double hydroxides or anionic clays. These compounds have a layered crystal structure composed of positively charged hydroxide layers and interlayers containing anions and water molecules. These compounds exhibit anion-exchange properties and can recover the layered crystal structure during rehydration.

The mesoporous material may be present in the sensor element in the dry state, in which case the material will require the addition of water, for example as water vapour present in the gas stream. Alternatively, the mesoporous material may be present with water in a saturated or partially-saturated state.

The thickness of the mesoporous material will determine the response of the sensor to changes in the composition of the gas stream in contact with the mesoporous layer. To minimize internal resistance within the sensor element, it is preferred to use an ultra thin mesoporous layer.

The mesoporous material may comprise a binder, in particular a conductive (ion exchanger type) binder. Suitable conductive binders include ionomers, a class of synthetic polymers with ionic properties. A particularly preferred group of ionomers are the sulphonated tetrafluoroethylene copolymers. An especially preferred ionomer from this class is Nafion®, available commercially from Du Pont. The sulphonated tetrafluroethylene copolymers have superior conductive properties due to their proton conducting capabilities. The pores in the mesoporous material allow movement of cations but the membranes do not conduct anions or electrons. The sulphonated tetrafluroethylene copolymers can be manufactured with various cationic conductivities. They also exhibit excellent thermal and mechanical stability and are biocompatible, thus making them suitable materials for use in the controlled electrode coating.

In one particularly preferred embodiment, the layer extending between the electrodes comprises an ion exchange material, optionally an inert filler, and a mesoporous material. In this respect, references to a mesoporous material are to a material having pores in the range of from 1 to 75 nm, more particularly in the range of from 2 to 50 nm, as noted above. The mesoporous material provides a medium that is highly controllable and hydrates uniformly to provide a suitable medium for conduction to occur.

Suitable mesoporous materials for use in the particularly preferred sensor of the present invention, as described hereinbefore, are known in the art and commercially available, and include Zeolites. Zeolites are a particularly preferred component for inclusion in the ion exchange layer in the sensor of the present invention. One preferred zeolite is Zeolite 13X. Alternative mesoporous materials for use are Zeolite 4A or Zeolite P. The ion exchange layer may contain one or a combination of zeolite materials.

The granularity and thickness of the mesoporous material will determine the response of the sensor to changes in the composition of the gas stream in contact with the ion exchange layer. To minimize internal resistance within the sensor, it is preferred to use an ultra thin layer containing mesoporous material.

The mesoporous material is preferably dispersed in the ion exchange layer, most preferably as a fine dispersion. The mesoporous material is preferably dispersed as particles having a particle size in the range of from 0.5 to 20 μm, more preferably from 1 to 10 μm. The particles of mesoporous material are preferably finely dispersed in the ion exchange layer such that adjacent particles are generally at least one particle diameter apart, more preferably generally from at least 3 to 5 particles diameters apart. More highly dispersed arrangements may also be used, with particles up to 10 diameters apart, for example, if required.

The sensor comprising a layer of ion-exchange material with a sparse population of mesoporous material therein may be prepared using any suitable technique. In one preferred method, the ion-exchange/mesoporous material layer is applied in a two-stage process. In the method, the particles of mesoporous material are applied first, for example by contacting the sensor to be coated with a suspension of mesporous particles of the desired dispersion in a suitable solvent, such as an alcohol. The solvent is removed, for example by evaporation, leaving a layer of dispersed mesoporous particles. Other techniques to deposit the particulate mesoporous material onto the surface of the sensors may also be used. Examples of other techniques include: dry aerosol deposition, spray pyrolysis, and screen printing. More complex techniques may also be employed, such as: in-situ crystal growth, hydrothermal growth, sputtering, autoclaving, and the like.

Thereafter, a layer of ion-exchange material may be applied to the required thickness. This may be accomplished by dispensing the required volume of ion-exchange in a suitable solvent onto the layer of dispersed mesoporous material. The solvent is then allowed to evaporate, leaving the required layer of ion-exchange material containing the mesoporous particles retained therein in a highly dispersed arrangement.

In one embodiment, the mesoporous material is applied to the electrodes as a suspension of particles in a suitable solvent, with the solvent being allowed to evaporate to leave a fine dispersion of particles over the electrodes. Ion exchange material is then applied over the mesoporous dispersion. The mesoporous material is preferably applied in a concentration of from 0.01 to 1.0 g, as a uniform suspension in 10 ml of solvent, into which the electrode assembly is dipped one or more times. More preferably, the mesporous material is applied in a concentration of from 0.05 to 0.5 g per 10 ml of solvent, especially about 0.1 g per 10 ml of solvent. Suitable solvents for use in the application of the mesoporous material are known in the art and include alcohols, in particular methanol, ethanol and higher aliphatic alcohols.

The dispersion of the mesoporous particles on the sensor element may be controlled by varying the concentration of the suspension of the particles and by the number and nature of contacts between the suspension and the sensor element.

It has been found that the sparse population of mesoporous particles within the (continuous) ion exchange film affords the highest discrimination towards the detection of target species in the gas stream, in particular water vapour. In particular, it has been found that a sensor having a layer of ion-exchange material comprising zeolite particles dispersed therein, as described above, is particularly sensitive to changes in the concentration of water vapour in the gas stream. In this way, the sensor may be used with a very high specificity to the detection of water vapour and a measurement of the water vapour concentration. Examination under a scanning electron microscope (SEM) of a preferred arrangement reveals a density of mesoporous particles such that each particle is, on average, distanced several body diameters, in particular from 1 to 5 body diameters, more preferably from 1 to 3 body diameters, away from the nearest neighbour.

It has also been found that thick films of ion exchange material degrade the performance of the sensor, as do thick continuous coats of the mesoporous material. In other words, it is the combination of a thin ion exchange layer and sparse, population of mesoporous particles that performs best.

A further sensor element embodiment comprises a solid electrolyte precursor extending between and in contact with the working electrode and the counter electrode; whereby the gas stream may be caused to impinge upon the solid electrolyte precursor such that the water vapour in the gas stream at least partially hydrates the precursor to form an electrolyte in electrical contact with the working electrode and the counter electrode.

In the context of the present invention, the term ‘solid electrolyte precursor’ is a reference to a material that is in the solid phase under the conditions prevailing during the use of the sensor element and that can react with (or be hydrated by) water vapour in the gas stream to reconstitute a hydrous electrolyte, allowing current to flow between the working electrode and counter electrode.

The solid electrolyte precursor comprises a ligand, preferably an organic ligand (hereafter denoted as ‘L’), which is capable of forming a complex with a metal ion (hereafter denoted as ‘M’) to form an organometallic complex. Within the electrolyte, the organic ligand is capable of dissociation according to the following equations:

LH₂ [symbol] LH⁻+H⁺

LH⁻ [symbol] L²⁻+H⁺

A wide range of ligands and metal ions may be employed in the organometallic complex of the solid electrolyte precursor. Preferred organic compounds for use as the ligand are amines, in particular diamines, such as diaminopropane, and carboxylic acids, especially dicarboxylic acids. The metal ions are preferably ions of Group VIII of the Periodic Table of the Elements (as provided in the Handbook of Chemistry and Physics, 62^{nd} edition, 1981 to 1982, Chemical Rubber Company). Suitable metals include copper, lead and cadmium.

The solid electrolyte precursor preferably also comprises a salt. Metal halide salts are preferred, in particular sodium and potassium halides, especially chlorides.

The specific choice and combination of metal ions and organic ligands may be theoretically calculated using principles of equilibrium (speciation) chemistry. The principle determinand is that the ligand should have a low pK_{b}. As noted above, a preferred class of ligand is the diamines, for example, propanediamine, ethylenediamine and various substituted diamines. The performance of the sensor is dependant on the choice and concentration of metal/ligand pairs and the optimum precursor composition may be found by routine experimentation.

As noted above, the active layer of the sensor element is preferably a thin layer, with extremely or ultra-thin layers being preferred, in particular in the case of an active layer comprising an ion exchange material. Preferably, the active layer has a thickness of from 1 to 1000 nm, more preferably from 5 to 500 nm, especially from 10 to 100 nm. In some cases, the thickness of the active layer may be of the same order and substantially the same as the diameter of particles retained in the layer, such as the particles of mesoporous or zeolite material.

A particularly preferred composition for the solid electrolyte precursor comprises copper, propanediamine and potassium chloride. One preferred composition has these components present in the following amounts: 4 mM copper, 10 mM propanediamine, and 0.1M potassium chloride as base electrolyte.

It will be appreciated by those skilled in the art that there are a considerable range and combination of other metals, ligands, and base electrolytes.

The solid electrolyte precursor may be prepared from a solution of the constituent components in a suitable solvent. Water is a most convenient solvent. The solvent is removed by drying and evaporation, to leave the solid electrolyte precursor. Evaporation of the solvent may be assisted by blowing a gas stream, such as air or nitrogen, across the surface of the drying precursor.

The sensor element comprises a first or working electrode and a second or counter electrode. Such a two-electrode construction is known in the art. The electrodes may comprise any suitable metal or alloy of metals, with the proviso that the electrode does not react with the active layer or any of the substances present in the gas stream. Preference is given to metals in Group VIII of the Periodic Table of the Elements (as provided in the Handbook of Chemistry and Physics, 62^{nd} edition, 1981 to 1982, Chemical Rubber Company). Other suitable metals include copper, silver and gold. Preferably, each electrode is prepared from copper, gold or platinum. Carbon or carbon-containing materials may also be used to form the electrodes.

The electrodes may have any suitable shape and configuration. Suitable forms of electrode include points, lines, rings and flat planar surfaces. The effectiveness of the sensor element can depend upon the particular arrangement of the electrodes and may be enhanced in certain embodiments by having a very small path length between the adjacent electrodes. This may be achieved, for example, by having each of the first and second electrodes comprise a plurality of electrode portions arranged in the form of an array of interdigitated electrode portions, in particular arranged in a concentric or rectangular pattern.

The electrodes are preferably oriented as close as possible to each other, to within the resolution of the manufacturing technology. The first and second electrode can be between 10 to 1000 microns in width, preferably from 50 to 500 microns. The gap between the first and second electrodes can be between 20 and 1000 microns, more preferably from 50 to 500 microns. The optimum track-gap distances are found by routine experiment for the particular electrode material, geometry, configuration, and substrate under consideration. In a preferred embodiment the optimum first (or working) electrode track widths are from 50 to 250 microns, preferably about 100 microns, and the second (or counter) electrode track widths are from 50 to 750 microns, preferably about 500 microns. The gaps between the first and second electrodes are preferably about 100 microns.

The first electrode and second electrode may be of equal size. However, in one preferred embodiment, the surface area of the second or counter electrode is greater than that of the first or working electrode to avoid restriction of the current transfer. Preferably, the counter electrode has a surface area at least twice that of the working electrode. Higher ratios of the surface area of the counter electrode and working electrode, such as at least 3:1, preferably at least 5:1 and up to 10:1 may also be employed. The thickness of the electrodes is determined by the manufacturing technology, but has no direct influence on the electrochemistry. The magnitude of the resultant electrochemical signal is determined principally by exposed surface area, that is the surface area of the electrodes directly exposed to and in contact with the gaseous stream. Generally, an increase in the surface area of the electrodes will result in a higher signal, but may also result in increased susceptibility to noise and electrical interference. However, the signals from smaller electrodes may be more difficult to detect.

In its simplest form, the sensor element consists of the electrodes in combination with the active layer. For example, the electrodes may be applied to the surface of the active material or encapsulated within or underneath the active material.

In a preferred embodiment, the sensor element comprises an inert support, upon which is deposited the active layer. The inert substrate may be any suitable material, with the proviso that it does not react or interact with the coating, the electrodes or the components in the gas stream to be analysed. Suitable inert substrates include glass, polymers, ceramics and the like. The use of an inert substrate offers the advantage of providing strength and rigidity to the sensor assembly. In addition, the inert substrate allows the thickness of the active coating to be reduced and the path length of the gas components entering the coating and the conductive path between the electrodes to be more closely controlled. This in turn provides for a sensor that is robust, more accurate and more responsive.

In one arrangement, the active material is deposited as a coating on top of the electrodes applied to the substrate layer. An alternative arrangement is to have at least one of the electrodes applied directly to the surface of the inert substrate and the active material applied as a layer over both the electrode and substrate. In this way at least one or both of the electrodes is disposed between the active layer and the inert substrate.

To improve the electrical insulation of the electrodes, the portions of the electrodes that are not disposed to be in contact with the gaseous stream (that is the non-operational portions of the electrodes) may be coated with a dielectric material, patterned in such a way as to leave exposed the active portions of the electrodes.

While the sensor operates well with two electrodes, as hereinbefore described, arrangements with more than two electrodes, for example including a third (or reference) electrode, as is well known in the art, may be employed. The use of a reference electrode provides for better potentiostatic control of the applied voltage, or the galvanostatic control of current, when the “iR drop” between the counter and working electrodes is substantial. Dual 2-electrode and 3-electrode cells may also be employed.

A further electrode, disposed between the counter and working electrodes, may also be employed. The temperature of the gas stream may be calculated by measuring the end-to-end resistance of the further electrode. Such techniques are known in the art.

The electrodes of the sensor element of the present invention may be formed by any suitable technique. One preferred technique comprises printing the electrode material in the form of a thick film screen printing ink onto the substrate. The ink consists of four components, namely the functional component, a binder, a vehicle and one or more modifiers. In the case of the present invention, the functional component forms the conductive component of the electrode and comprises a fine powder of one or more of the aforementioned metals used to form the electrode.

The binder holds the ink together on the substrate and merges with the substrate during high temperature firing. The vehicle acts as the carrier for the powders and comprises both volatile components, such as solvents, and non-volatile components, such as polymers. Both the binder and vehicle materials evaporate during the early stages of drying and firing respectively. The modifiers comprise small amounts of additives, which are active in controlling the behaviour of the inks before and after processing.

Screen printing requires the ink viscosity to be controlled within limits determined by rheological properties, such as the amount of vehicle components and powders in the ink, as well as aspects of the environment, such as ambient temperature.

The printing screen may be prepared by stretching stainless steel wire mesh cloth across the screen frame, while maintaining high tension. An emulsion is then spread over the entire mesh, filling all open areas of the mesh. A common practice is to add an excess of the emulsion to the mesh. The area to be screen printed is then patterned on the screen using the desired electrode design template.

The squeegee is used to spread the ink over the screen. The shearing action of the squeegee results in a reduction in the viscosity of the ink, allowing the ink to pass through the patterned areas onto the substrate. The screen peels away as the squeegee passes. The ink viscosity recovers to its original state and results in a well defined print. The screen mesh is critical when determining the desired thick film print thickness, and hence the thickness of the completed electrodes.

The mechanical limit to downward travel of the squeegee (downstop) should be set to allow the limit of print stroke to be 75-125 μm below the substrate surface. This will allow a consistent print thickness to be achieved across the substrate whilst simultaneously protecting the screen mesh from distortion and possible plastic deformation due to excessive pressure.

To determine the print thickness the following equation can be used:

T_{w}=(Tₘ×Aₒ)+Tₑ

where: T_{w}=Wet thickness (μm);
Tₘ=mesh weave thickness (μm);
A₀=% open area;
Tₑ=Emulsion thickness (μm).

After the printing process the sensor element needs to be levelled before firing. The levelling permits mesh marks to fill and some of the more volatile solvents to evaporate slowly at room temperature. If all of the solvent is not removed in this drying process, the remaining amount may cause problems in the firing process by polluting the atmosphere surrounding the sensor element. Most of the solvents used in thick film technology can be completely removed in an oven at 150° C. when held there for 10 minutes.

Firing is typically accomplished in a conveyor belt furnace. Firing temperatures vary according to the metal powder and ink chemistry. Most commercially available systems fire at 850° C. peak for 10 minutes. Total furnace time is 30 to 45 minutes, including the time taken to heat the furnace and cool to room temperature. Purity of the firing atmosphere is critical to successful processing. The air should be clean of particulates, hydrocarbons, halogen-containing vapours and water vapour.

Alternative techniques for preparing the electrodes and applying them to the substrate or inert support, if present, include spin/sputter coating and visible/ultraviolet/laser photolithography. In order to avoid impurities being present in the electrodes, which may alter the electrochemical performance of the sensor, the electrodes may be prepared by electrochemical plating (“electroplating”). In particular, each electrode may be comprised of a plurality of layers applied by different techniques, with the lower layers prepared using one of the aforementioned techniques, such as screen-printing, and the uppermost or outer layer or layers being applied by electrochemical plating using a pure metal salt, such as gold chloride for the electroplating of gold metal.

In a further aspect, the present invention provides a method of monitoring the respiration pattern of a subject, the method comprising:

contacting a gas stream exhaled by the subject with the sensor element of an electrochemical sensor assembly, thereby generating an signal from the sensor element in response to changes in the concentration of water vapour in the exhaled gas stream;

processing the electrical signal to determine the respiration pattern of the subject; and

generating a display of information relating to the respiration pattern for a user.

The method may be used to monitor a range of aspects of the respiration pattern of the subject, in particular the rate of respiration and/or the depth of respiration.

The method of the present invention employs an electrochemical sensor to monitor changes to the concentration of water vapour in the exhaled gas stream. The method preferably comprises contacting both inhaled and exhaled gas streams of the subject with the sensor element, in particular during tidal breathing of the subject.

The sensor assembly and sensor element may be of the general or specific kind described hereinbefore. In operation, the conductivity of the electrical path between the electrodes of the sensor element changes in response to changes in the concentration of water vapour in the impinging gas stream.

The variation in the electrical conductivity of the electrical path between the electrodes may be determined by applying a voltage to the first and second electrodes. The voltage may be applied in a continuous (dc) manner or in an ac, intermittent or pulsed form. The voltage, when applied, may be a constant voltage or may cycle between a lower (rest) voltage and a higher voltage.

The method requires that an electric potential is applied across the electrodes and conductivity estimated by the measurement of current passing between the electrodes. In one simple configuration, a voltage is applied to the counter electrode, while the working electrode is connected to earth (grounded). In its simplest form, the method applies a single, constant potential difference across the working and counter electrodes. Alternatively, the potential difference may be varied against time, for example being pulsed or swept between a series of potentials. In one embodiment, the electric potential is pulsed between a so-called ‘rest’ potential, at which no reaction occurs, and a reaction potential.

In operation, a linear potential scan, sinusoidal waveforms, multiple voltage steps or one discrete potential pulse are applied to the working electrode, and the resultant Faradaic reduction current is monitored as a direct function of the dissolution of water molecules in the coating bridging the electrodes.

The current that passes between the counter and working electrodes is converted to a voltage using a resistor, R, and recorded as a function of the water vapour concentration in the gaseous stream. The measured current in the sensor element is usually small. As a result of the small current flow, careful attention to electronic design and detail may be necessary. In particular, special “guarding” techniques may be employed. Ground loops need to be avoided in the system. This can be achieved using techniques known in the art. The sensor responds faster by pulsing the potential between two voltages, a technique known in the art as ‘Square Wave Voltammetry’. Measuring the response several times during a pulse may also be used to assess the impedance of the sensor.

The shape of the transient response can be simply related to the electrical characteristics (impedance) of the sensor in terms of simple electronic resistance and capacitance elements. By careful analysis of the shape, the individual contributions of resistance and capacitance may be calculated. Such mathematical techniques are well known in the art. Capacitance is an unwanted noisy component resulting from electronic artefacts, such as charging, etc. The capacitive signal can be reduced by selection of the design and layout of the electrodes in the sensor. Increasing the surface area of the electrodes and increasing the distance between the electrodes are two major parameters that affect the resultant capacitance. The desired Faradaic signal resulting from the passage of current due to reaction between the electrodes may be optimized, by experiment. Measurement of the response at increasing periods within the pulse is one technique that can preferentially select between the capacitive and Faradaic components, for instance. Such practical techniques are well known in the art.

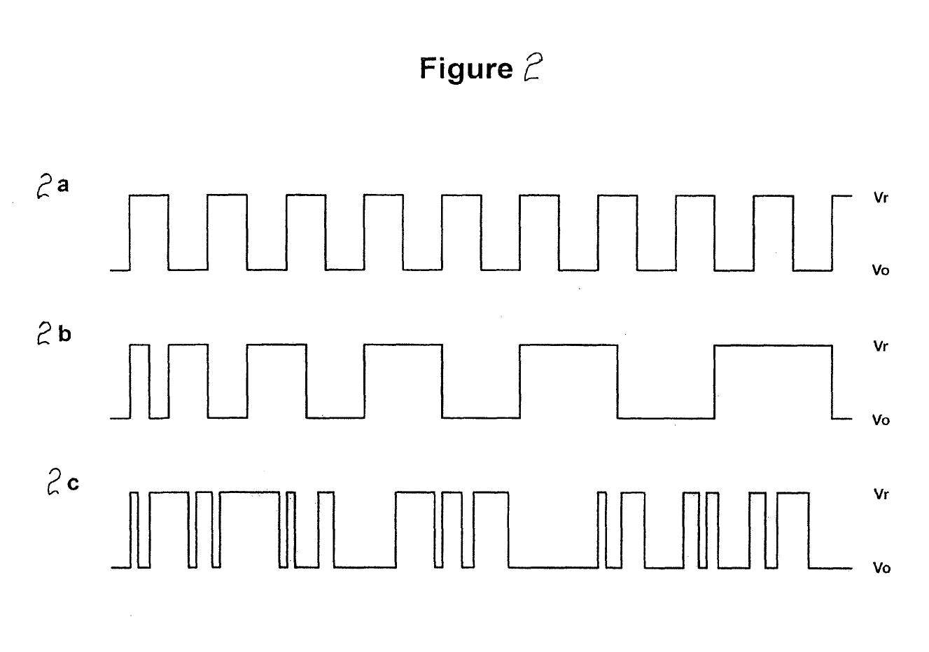

The potential difference applied to the electrodes of the sensor element may be alternately or be periodically pulsed between a rest potential and a reaction potential, as noted above. FIG. 2 shows examples of voltage waveforms that may be applied. FIG. 2a is a representation of a pulsed voltage signal, alternating between a rest potential, V₀, and a reaction potential V_{R}. The voltage may be pulsed at a range of frequencies, typically from sub-Hertz frequencies, that is from 0.1 Hz, up to 10 kHz. A preferred pulse frequency is in the range of from 1 to 500 Hz. Alternatively, the potential waveform applied to the counter electrode may consist of a “swept” series of frequencies, represented in FIG. 2b. A further alternative waveform shown in FIG. 2c is a so-called “white noise” set of frequencies. The complex frequency response obtained from such a waveform will have to be deconvoluted after signal acquisition using techniques such as Fourier Transform analysis. Again, such techniques are known in the art.

One preferred voltage regime is 0V (“rest” potential), 250 mV (“reaction” potential), at a 20 Hz pulse frequency.

It is an advantage of the sensor element of the preferred embodiments of the present invention that the electrochemical reaction potential is approximately +0.2 volts, which avoids many (if not all) of the possible competing reactions that would interfere with the measurements, such as the reduction of other metal ions (such as copper ions) and atmospheric oxygen.

The sensor assembly and method of the present invention are of use in monitoring and determining the respiratory rate of breathing of a subject, in particular a human patient or an animal. The method and sensor assembly are particularly suitable for analyzing tidal concentrations of water vapour in the exhaled breath of a subject to diagnose or monitor a variety of respiratory conditions. The sensor assembly is particularly useful for applications requiring fast response times, for example personal respiratory monitoring of patients undergoing surgery. Water vapour measurements can be applied generally in the field of respiratory medicine, airway diseases, both restrictive and obstructive, airway tract disease management, and airway inflammation. The present invention finds particular application in the field of monitoring and management of airway diseases (such as asthma and COPD). In particular, due to the versatility of the method and speed of response that may be achieved using the sensor and method of the present invention, the results may be used to provide an early alert to the onset of respiratory disease or illness.

The sensor element and sensor assembly of the present invention are easily fabricated from low-cost materials and are adaptable for use in various medical environments, and for the monitoring of various medical conditions. The respiratory rate sensor and apparatus is particularly suitable for use in hospital and primary healthcare, especially for critical care patients.

In one preferred arrangement, the sensor element of the sensor assembly of the present invention is arranged to be disposed in the nasal passages or cannulae of the subject. In this way, the problems of wearing a mask are avoided.

In addition, the sensor assembly of the present invention may further comprise sensor elements responsive to other components in the gas stream exhaled by the subject. Examples include oxides of nitrogen, oxygen and carbon dioxide. In this way, a plurality of sensor elements may be used to generate signals for processing by a single or plurality of processors, allowing the user to obtain a respiratory profile for the subject. As noted hereinbefore, the sensor elements required are preferably arranged to be disposed within the nasal passages of the subject.

The sensor assembly of the present invention may also comprise means for measuring the cardiac output of the subject, for example the pulse rate of the subject and/or blood pressure and the like. Means for such measurement are known in the art and include pulse oximetry. It has now been found that the nasal septum is an advantageous location on the body of a subject to measure pulse rate, allowing such measuring means to be conveniently combined with the electrochemical sensor elements of the sensor assembly.

As noted above, the sensor assembly of the present invention finds use in the monitoring of a subject suffering from sleep apnea. In particular, as also discussed, the sensor assembly may be used to provide an indication of the imminent start of an apneic event. In many cases, an apneic event is preceded by a period of erratic breathing by the subject, during which the rate and/or depth of respiration become erratic and irregular. In such a case, the sensor assembly may be used to identify when an apneic event is likely to occur. If left, the subject will experience the apneic event, during which respiration will cease.

In its simplest form, the sensor assembly of the present invention may, therefore be used, as a tool in the diagnosis of sleep apnea. Currently, the diagnosis of sleep apnea is complicated, requiring the subject to undergo a series of tests and analyses, typically in a sleep clinic, employing an extensive range of equipment. Typically, the subject is monitored throughout one or more night's sleep and the breathing patterns of the subject observed and measured to properly diagnose the condition of sleep apnea. This approach is both very time consuming and costly, in terms of the equipment and facilities required for such an extensive investigation. The sensor assembly of the present invention avoids the need for such extensive facilities and such a disruptive or invasive procedure. In particular, the sensor assembly of the present invention may be provided to a subject suspected of experiencing sleep apnea for use at home. This arrangement also has the advantage of providing a better analysis of the subject than may be obtained from the subject in a strange or unfamiliar environment. It has been found that the data generated by the sensor assembly of the present invention relating to the respiration patterns of the subject, in particular relating to respiratory rate and depth of respiration, can provide an accurate indication of sleep apnea to a clinician. In particular, the respiration patterns of the subject for an entire night can be easily detected and monitored, showing in particular the periods of erratic respiration pattern preceding an apneic event and the event itself, during which the subject stops breathing for a period of time.

Advantageously, in one embodiment, the sensor assembly of the present invention may further comprise a means for changing the level of arousal of the subject. In this way, the sensor assembly may be used to detect the erratic breathing by the subject leading up to an apneic event and the subject stimulated or disturbed to prevent the event occurring. The means may be triggered by the detection of a period of erratic or irregular breathing and thus respond directly or indirectly to the signal data being generated by the sensor element of the assembly. The means for changing the level of arousal of the subject may sufficient to disturb the subject from their current state, but are preferably not sufficient to fully awaken the subject from sleep. In particular, the means are preferably suitable to change the level of arousal of the subject sufficient to bring them out of REM sleep patterns to a higher arousal level. The means for arousing the subject may be any suitable means and include, for example, mechanical devices to impart a mechanical movement or force to the subject. In one embodiment, the means entail providing the subject with a mild electric shock, sufficient to change their state of arousal to the required degree.

Further, the sensor assembly of the present invention finds use in the treatment of sleep apnea. It is known and widely practiced to treat a subject suffering from sleep apnea using a continuous positive airway pressure (CPAP) machine. The CPAP machine supplies the subject with a continuous stream of pressurized air, typically through a nasal pillow, nose mask or a full face mask. The air is delivered at sufficient pressure to hold the airways of the subject open, that is act as a splint, preventing the airways from closing and becoming obstructed. Depending upon the severity of the condition endured by the subject, the stream of air required to be delivered by the CPAP machine can be at a significant flowrate and pressure. This can lead to difficulties for the subject when this must be endured for long periods of time to prevent the onset of an apneic event. The sensor assembly of the present invention may be combined with a CPAP machine and the output from the sensor element and the processor used to control the CPAP machine. In particular, the sensor assembly can be used to identify an erratic respiration pattern symptomatic of an apneic event. When such an erratic pattern is detected, the sensor assembly may be used to activate the CPAP machine to deliver the air stream required to prevent the apneic event from taking place. Once a normal respiration pattern has been restored, the CPAP machine may then be turned off. In this way, the sensor assembly of the present invention may increase the acceptability and useability of a CPAP to sufferers of sleep apnea.

In addition to the uses in monitoring the respiration of a subject as an aspect of monitoring the subject, for example as part of a diagnosis routine or in monitoring the subject while under treatment, the sensor assembly and method of the present invention find use in the training of the subject.

First, the sensor assembly and method may be used by the subject to aid the training of the metabolism. Second, the sensor assembly and method may be used by the subject to train lung function. In both cases, the information displayed by the sensor assembly is used by the subject, either alone or under instruction from a medical practitioner, trainer or the like, to adjust patterns of behaviour in order to improve aspects of metabolism and lung function.

Embodiments of the present invention will now be described, by way of example only, having reference to the accompanying drawings, in which:

FIG. 1 is a typical graphical trace of the concentration of water vapour in the exhaled gas stream of a human subject shown plotted against the time of exhalation;

FIGS. 2a, 2b and 2c are voltage versus time representations of possible voltage waveforms that may be applied to the electrodes of the sensor element in the method of the present invention, as discussed hereinbefore;

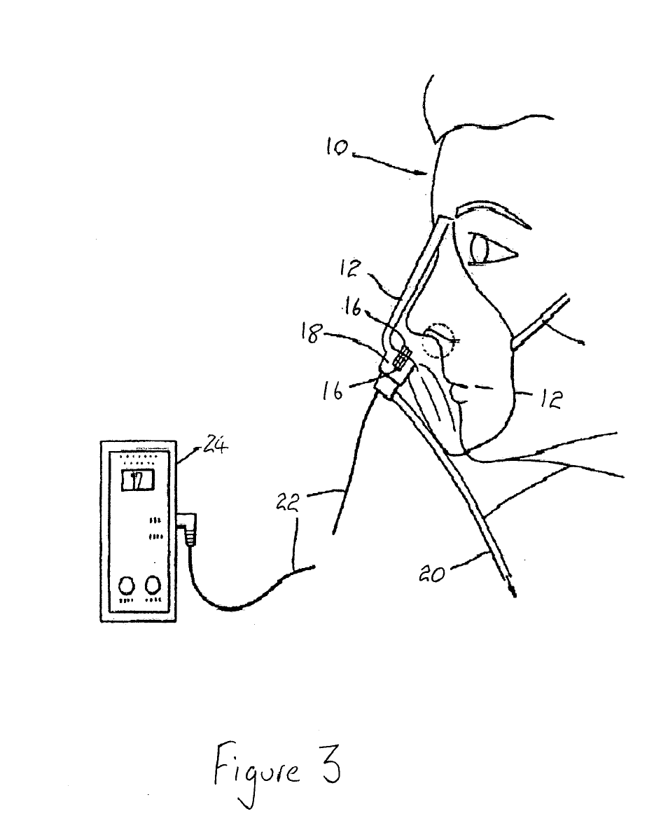

FIG. 3 is a perspective schematic view of a sensor assembly of a first embodiment of the present invention;

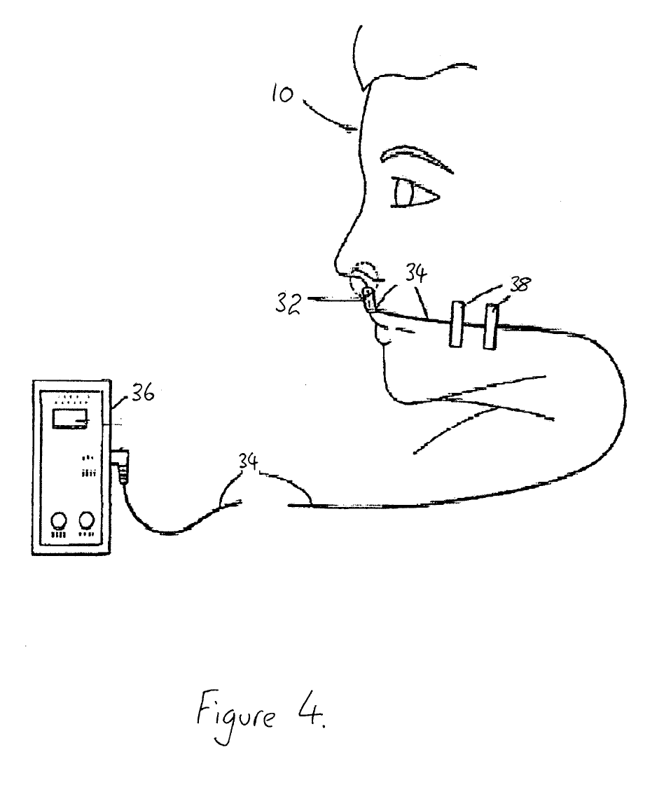

FIG. 4 is a perspective schematic view of a sensor assembly of a second embodiment of the present invention;

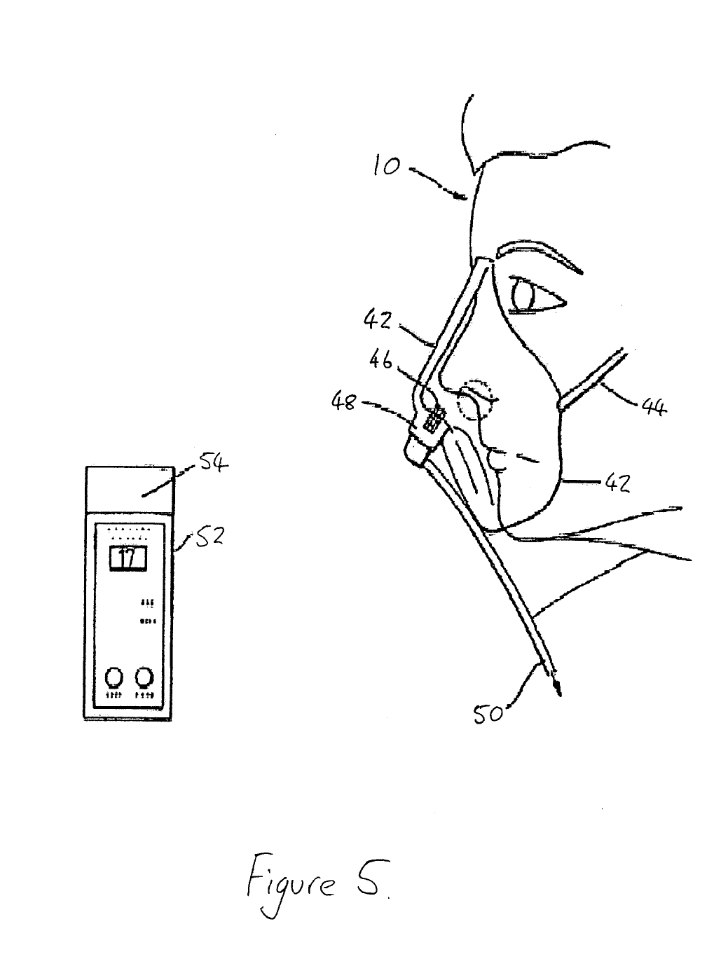

FIG. 5 is a perspective schematic view of a sensor assembly of a third embodiment of the present invention;

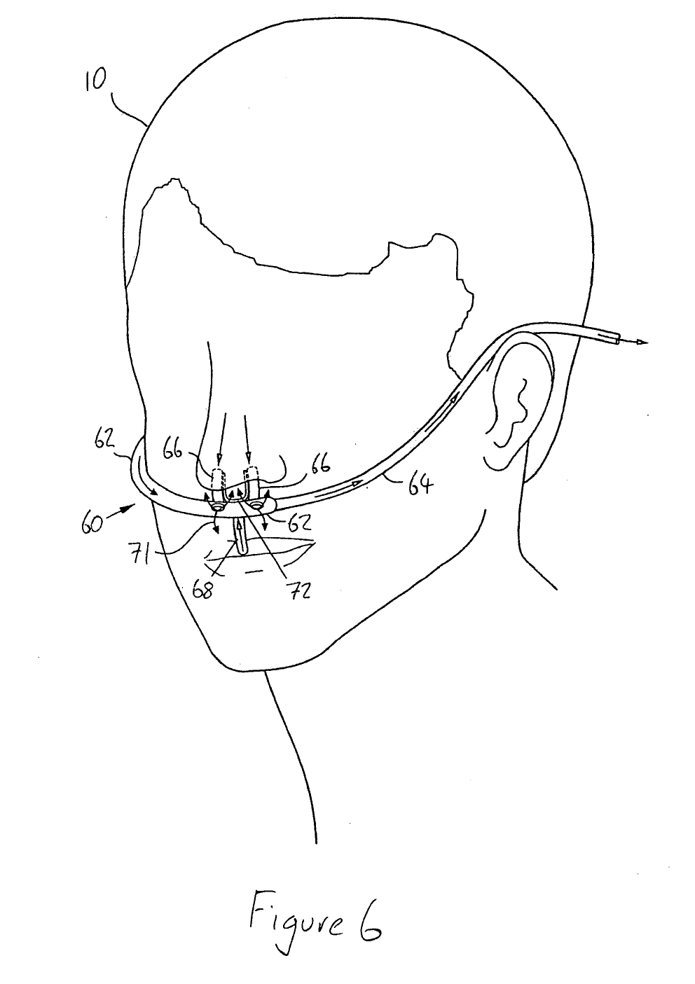

FIG. 6 is a schematic representation of a sensor assembly of the present invention incorporated into a standard oxygen delivery gas line;

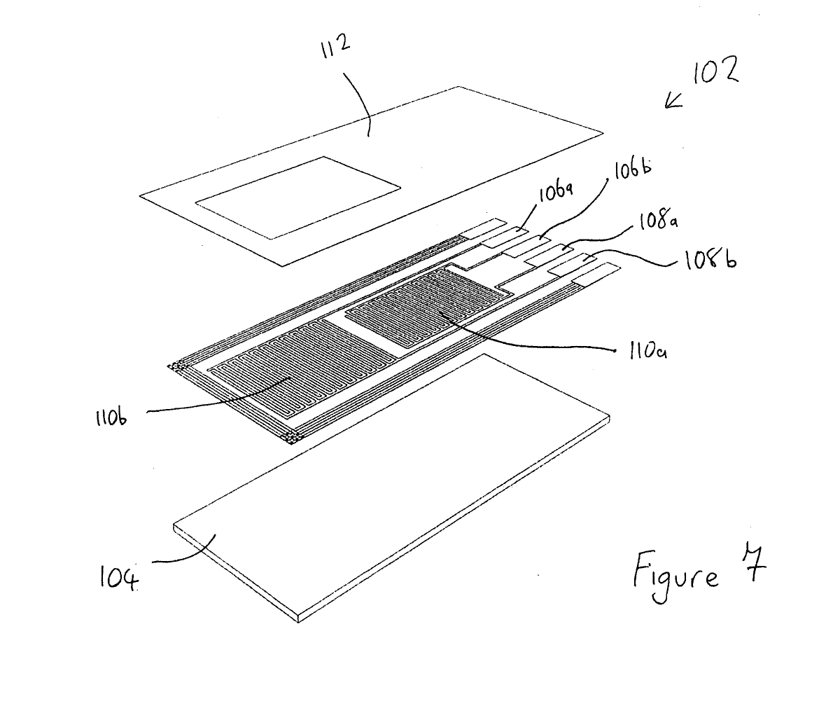

FIG. 7 is an exploded isometric sectional view of a sensor element according to the present invention;

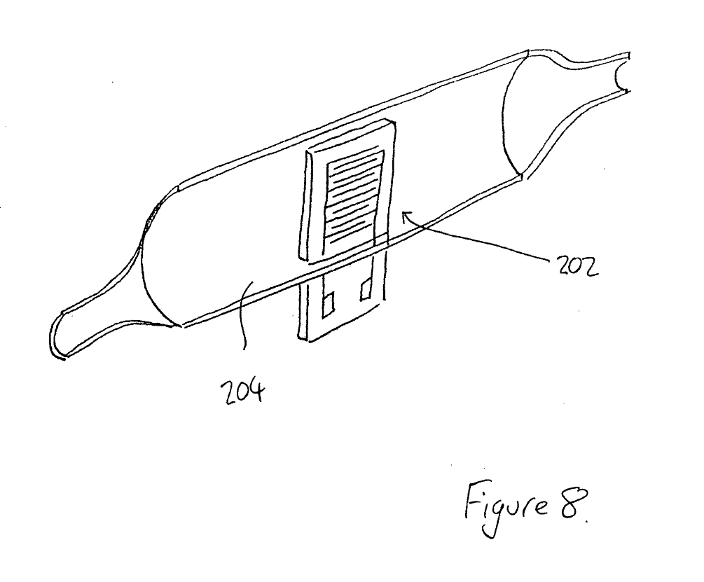

FIG. 8 is a schematic perspective view of the sensor element of FIG. 7 disposed within a conduit for the passage of inhaled and exhaled gases;

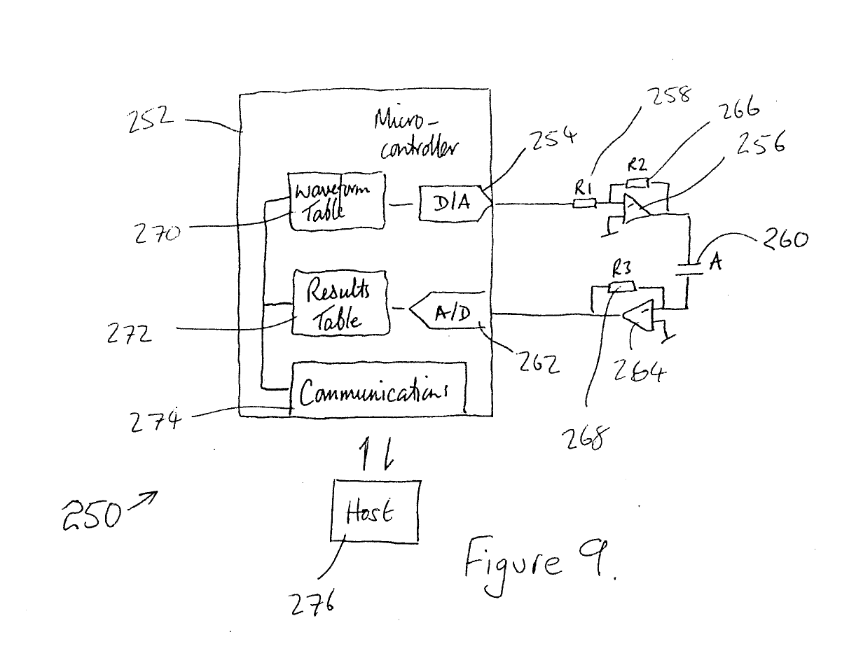

FIG. 9 is a diagrammatic representation of an embodiment of the sensor assembly of the present invention;

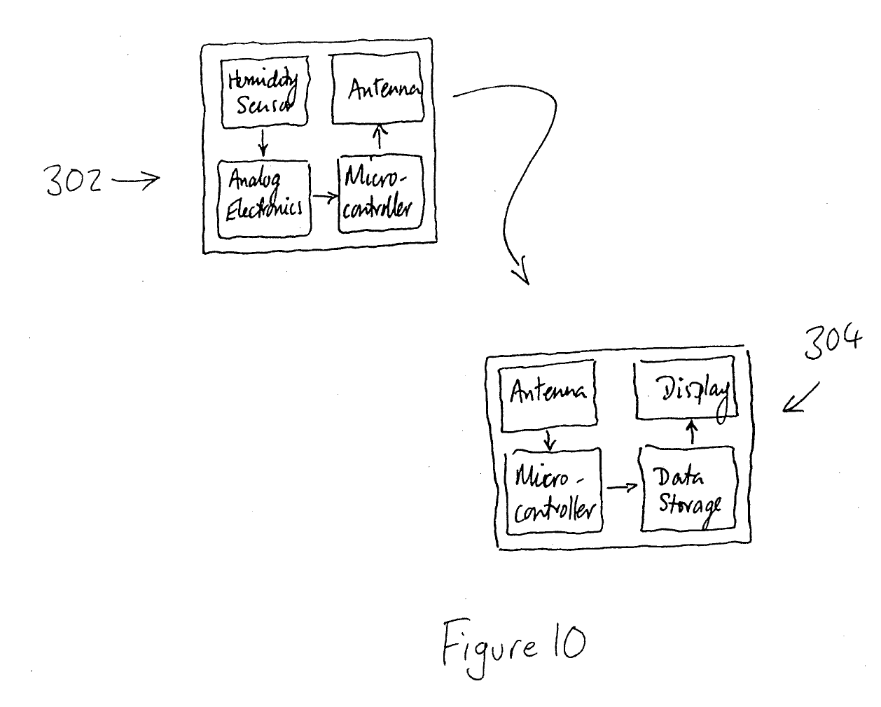

FIG. 10 is a schematic representation of the electrical components of an embodiment of the sensor assembly according to the present invention;

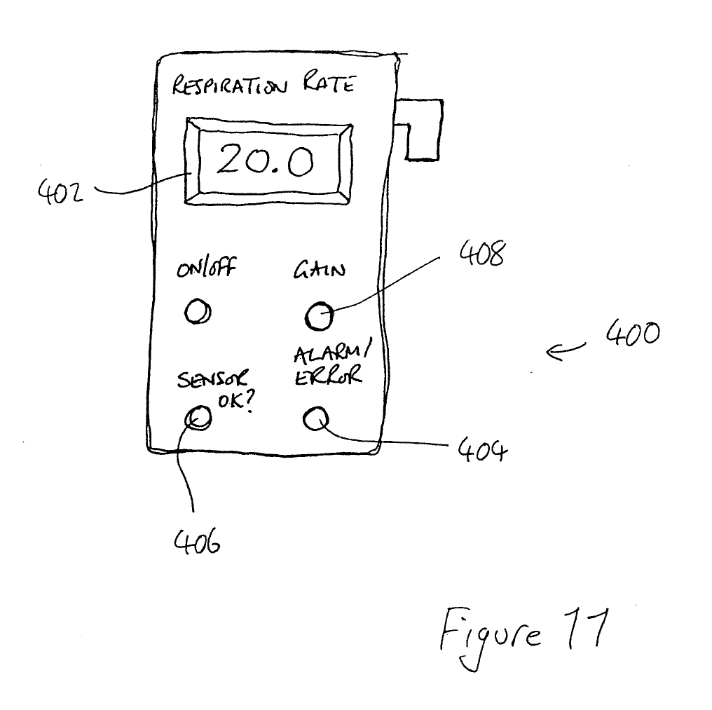

FIG. 11 is a schematic representation of a display unit for use in the sensor assembly of the present invention;

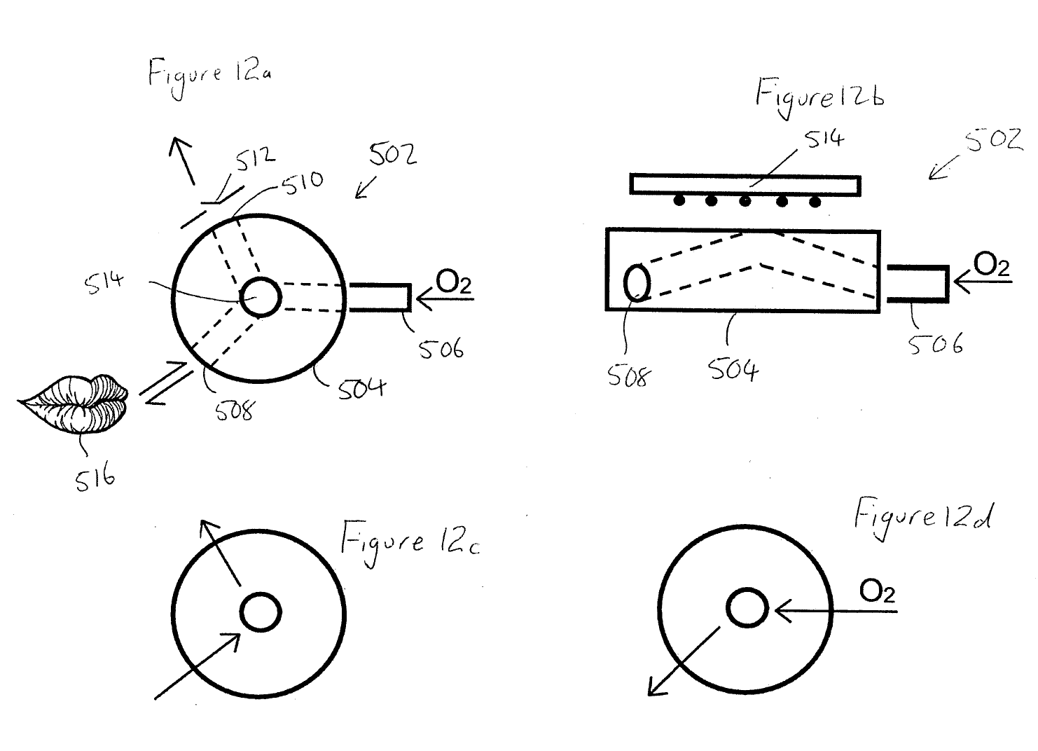

FIGS. 12a to 12d are schematic representations of a sensor element assembly with a function for drying the sensor element;

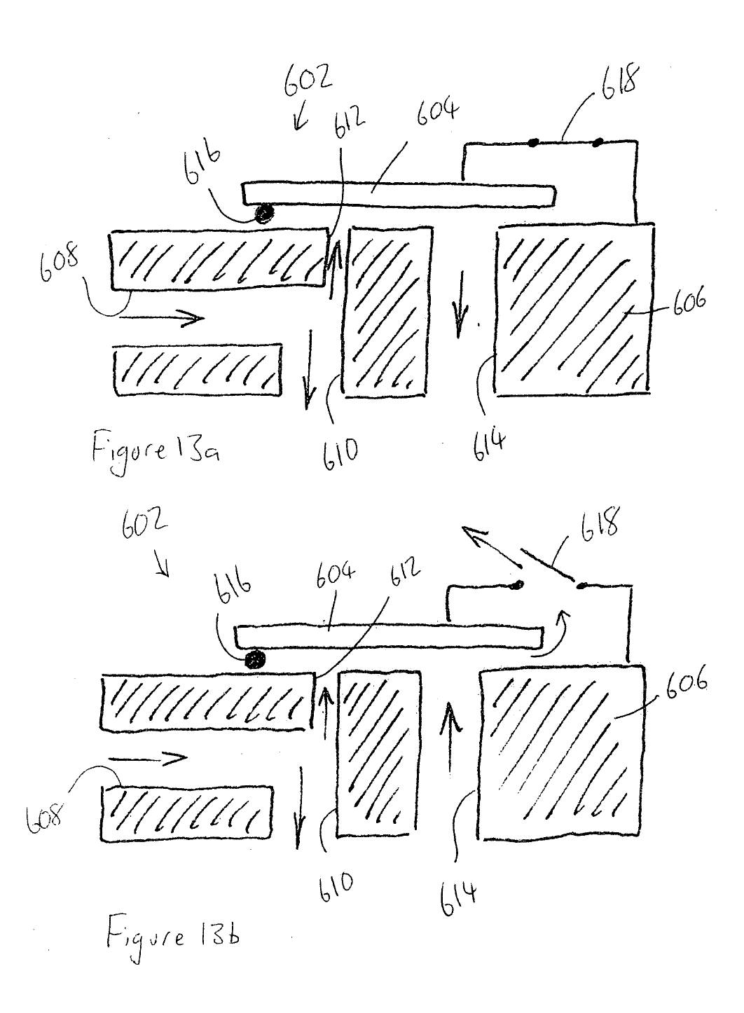

FIG. 13a is a schematic cross-sectional representation of a sensor element assembly with a function for drying the sensor element in the mode for inhalation by the subject;

FIG. 13b is a schematic cross-sectional representation of the sensor element assembly of FIG. 13a in the mode for exhalation by the subject;

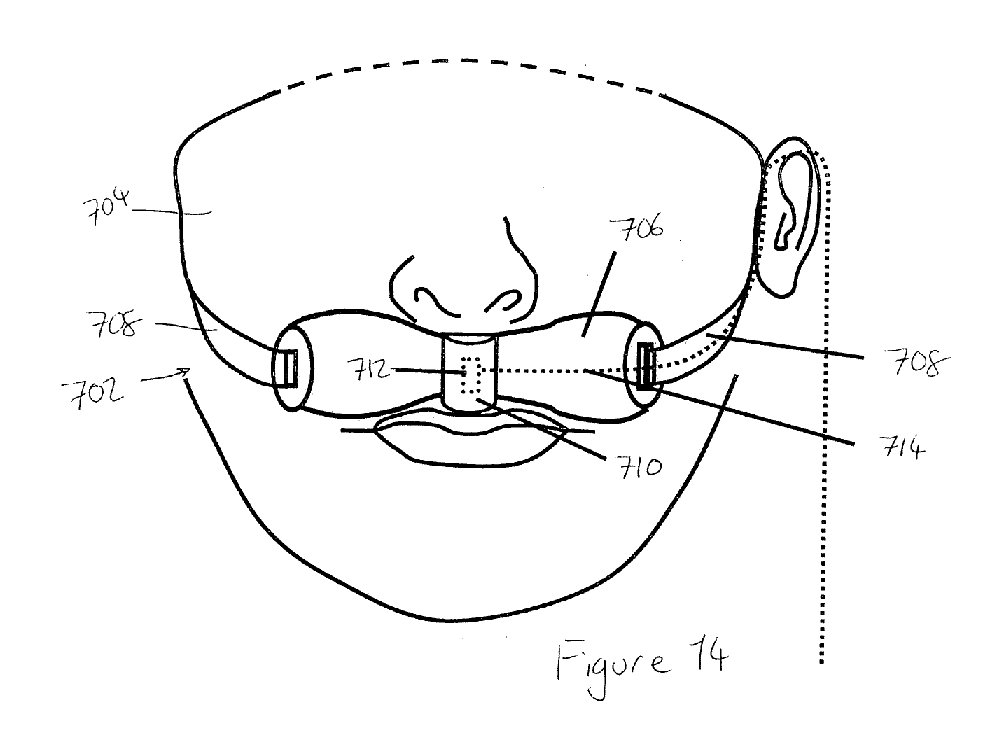

FIG. 14 is a frontal diagrammatical representation of a sensor element assembly of a further embodiment of the present invention in place on the head of a subject;

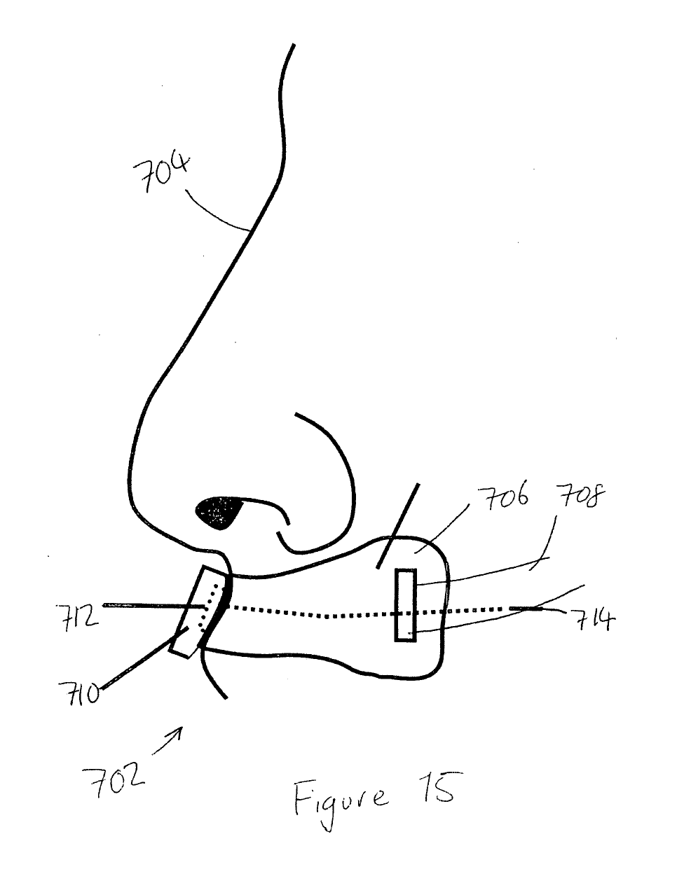

FIG. 15 is a side view of the sensor element assembly of FIG. 14;

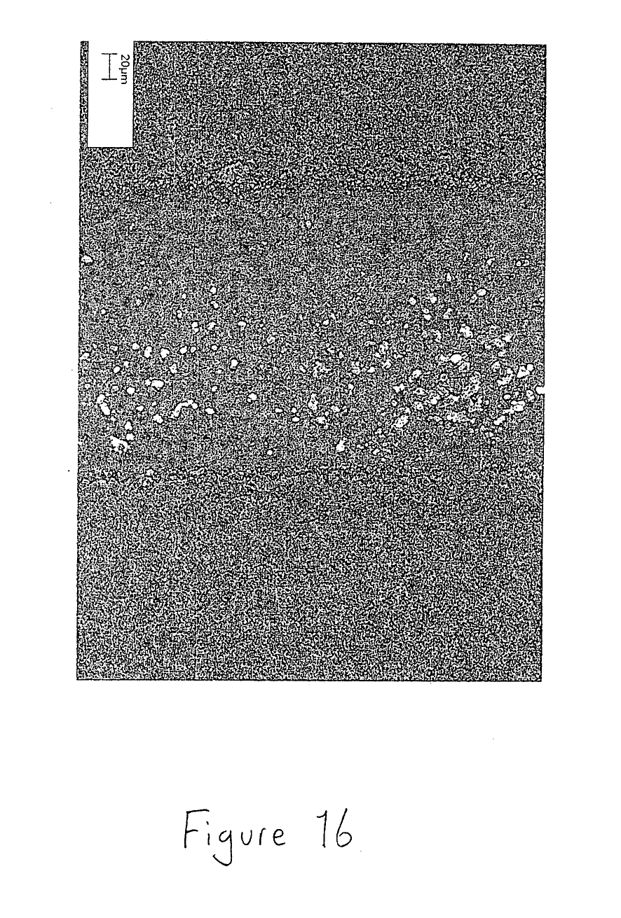

FIG. 16 is a scanning electron microscope (SEM) image of a portion of a sensor element showing a dispersion of microporous particles on an electrode, before application of a coating of ion exchange material; and

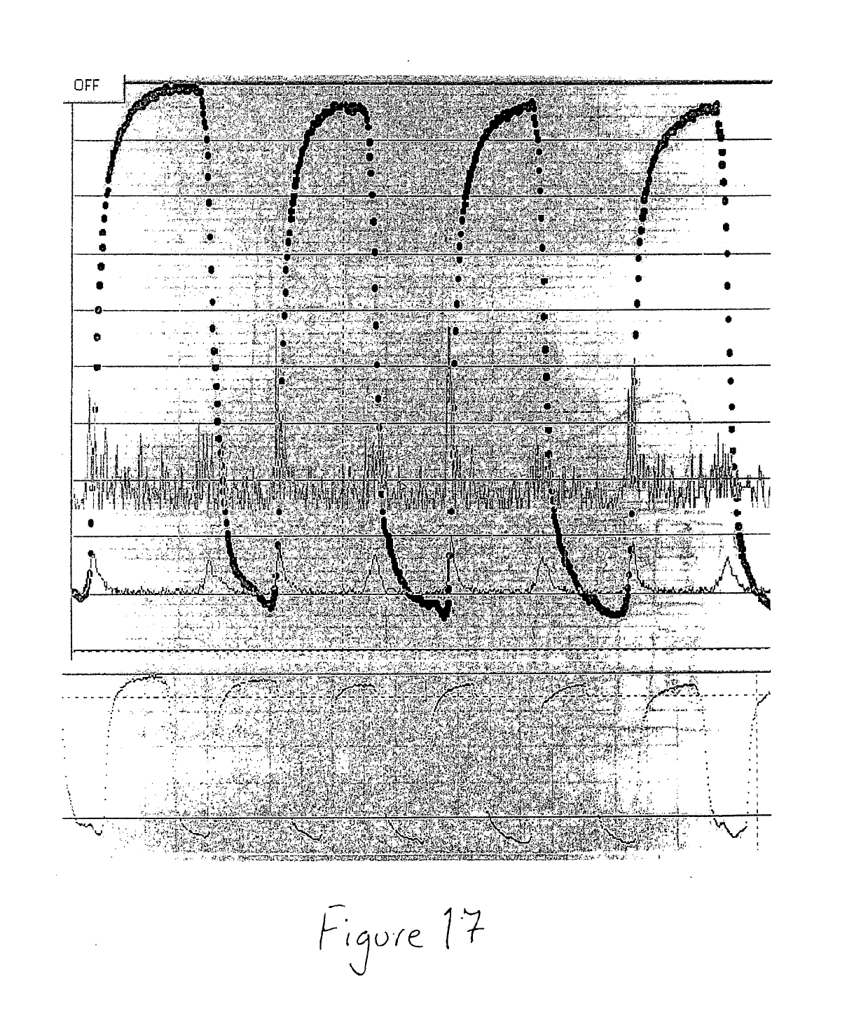

FIG. 17 is a graphical response obtained from the use of the sensor assembly shown' in FIG. 16 in a method according to the present invention.

Referring to FIG. 1, there is shown a graph of water vapour concentration plotted against time obtained from the measurement of the water vapour concentration in an exhaled gas stream throughout the, period of the exhalation. Similar traces are obtained with other gaseous components in the exhaled gas stream, most notably carbon dioxide, in which case the trace is known in the art as a ‘capnogram’.

The trace in FIG. 1 may be characterized as having points of inflection, indicated as points A, B, C, D and E, which generally separate four phases of the trace. Phase I is the volume of water vapour-free gas that is produced at the very start of exhalation by the subject. Phase II is the ascending phase, characterized by a rapid increase in the concentration of water vapour and represents the transition from water vapour-free gas to the early emptying portions of the lung. Phase III is the (alveolar) plateau phase and corresponds to the later emptying portions of the lungs, where the concentration of water vapour continues to slowly rise with time. Point D is generally indicated to be the end-tidal concentration of water vapour (PetH₂O). Phase IV of the trace is the final stage, where the water vapour concentration falls rapidly to that of the ambient gas composition.

Referring to FIG. 3, there is shown a subject 10 wearing a face mask 12 of largely conventional design retained by straps 14. The face mask 12 comprises a sensor element 16 according to the present invention and of the general configuration hereinbefore described. The sensor element 16 is disposed within an outlet channel 18 of the face mask, through which the gas stream exhaled by the subject 10 is caused to flow, before leaving through an exhaust line 20. The sensor element 16 is exposed to the gas stream of the subject as it is exhaled and leaves the mask. The sensor element 16 is connected by an electrical cable 22 to a processor and display device 24, the signals output from the sensor element being processed to generate a numerical display of the respiration rate of the subject.

Referring to FIG. 4, there is shown a subject 10 having a sensor element 32 attached to their upper lip. The sensor element 32 is according to the present invention and of the general configuration hereinbefore described. The sensor element 32 is disposed so as to be impinged by the gas stream exhaled by the subject 10 through the nostrils. The sensor element 32 is connected by an electrical cable 34 to a processor and display device 36, the signals output from the sensor element being processed to generate a numerical display of the respiration rate of the subject. The cable 34 is supported by portions of adhesive tape 38 adhered to a cheek of the subject 10.

Referring to FIG. 5, there is shown a subject 10 wearing a face mask 42 of largely conventional design retained by straps 44. The face mask 42 comprises a sensor element 46 according to the present invention and of the general configuration hereinbefore described. The sensor element 46 is disposed on an outlet channel 48 of the face mask, through which the gas stream exhaled by the subject 10 is caused to flow, before leaving through an exhaust line 50. The sensor element 46 is exposed to the gas stream of the subject as it is exhaled and leaves the mask. The sensor element 46 is connected to a processor and display device 52 by a wireless communications system 54, the signals output from the sensor element being processed to generate a numerical display of the respiration rate of the subject.

Referring to FIG. 6, there is shown a subject 10 wearing an oxygen supply device 60 of conventional design to supply oxygen to the nostrils of the subject. The supply device 60 comprises a feed line 62 for oxygen or oxygen-enriched air and an exhaust line 64 for exhaled gases. Tubes 66 are disposed to extend from the feed line 62 into the nostrils of the subject 10. A further tube 68 extends from the feed line 62 into the mouth of the subject. Holes in the feed line allow gases to leave the feed line 62 and enter the mouth or nose when the subject inhales. The subject 10 breathes normally, inhaling gases, indicated by arrows 71, from the feed line 62, with exhaled gases passing in the reverse direction through the feed tubes to the exhaust line. A sensor element 70 according to the present invention and of the general configuration hereinbefore described is disposed in the supply device 60 so as to be exposed to gas streams exhaled by the subject through either the mouth or nose. The tubes 66 are retained on either side of the septum of the subject by a clip 72.

Referring to FIG. 7, there is shown an exploded view of a sensor element, generally indicated as 102. The sensor element comprises a layer of inert substrate material 104. A set of electrodes comprising two pairs of working and counter electrodes 106a, 106b and 108a, 108b are disposed on a major surface of the inert substrate 104. Each of the working electrodes 106a, 106b and working electrodes 108a and 108b have portions extending in an interdigitated array 110a and 110b. A layer of active material 112 extends over the electrodes, the active material being responsive to changes in the concentration of water vapour in the gas stream impinging on the sensor element, such that the conductivity of the electrical path between the respective working and counter electrodes varies with changes in the water vapour concentration.

FIG. 8 shows a sensor element 202 of the general configuration shown in FIG. 7 mounted in a conduit 204, such that the sensor element 202 is in contact with gas streams passing through the conduit. The conduit may be used as a passage for inhaled and exhaled gas streams.

FIG. 9 is is a schematic representation of a microcontroller analog and digital interface for the electronic components of a sensor assembly of the present invention. The interface, generally indicated as 250 comprises a microcontroller 252 having a digital-to-analog (D/A) output 254 connected to a potentiostat circuit 256 through a resistor 258, in turn connected to the counter electrode of the sensor element 260. The input to the microcontroller 252 comprises an analog-to-digital (A/D) converter 262 connected through a current-to-voltage (i/E) converter 264 to the working electrode of the sensor element 260. The potentiostat circuit 256 and the current-to-voltage converter 264 are bridged by resistors 266 and 268, respectively. The microcontroller 252 has a range of memory devices and buffers, examples of which are shown in FIGS. 9 as 270 and 272. A communications port 274 is provided to allow the microcontroller 252 to communicate and exchange data with an external device, such as a host device 276.

An example of the functionality of the system of FIG. 9 is as follows:

The waveform applied to the sensor is digitally constructed as a table in the memory 272 of the microcontroller 252. Each value from the table is output by the digital-to-analog (D/A) 254 to the potentiostat circuit 256 sequentially at a rate appropriate to the frequency of the applied waveform. The attentuation of the input voltage waveform is controlled by the ratio of resistors 258 and 266. The output from the potentiostat circuit 256 is applied to the counter electrode of the sensor 260. The corresponding working electrode is connected to the current-to-voltage (i/E) converter 264. The gain of the current-to-voltage stage amplifier is controlled by the resistor 268. The output from the current-to-voltage converter 264 is digitized by the A/D converter 262 at the same rate as the output waveform, and held (buffered) in a results table 272 corresponding with and equivalent to the output table 270. The microcontroller software is capable of automatically comparing the input and output tables, or transmitting the data to the external host 276 for later manipulation.

Referring to FIG. 10, there is shown a schematic representation of one arrangement of electrical components of the sensor assembly of the present invention. In particular, the sensor assembly comprises a first sub-assembly 302 comprising a sensor element to measure the humidity of the gas stream being monitored and associated electrical components to generate an electrical signal. The signal is communicated, for example by a cable or by wireless means, to a second sub-assembly 304, in which the signals are processed and a display generated for providing information to the user.

An example of a display device 400 for providing information to a user is shown in FIG. 11. As shown, the display device comprises a numerical display 402, an alarm indicator 404, and a means 406 for testing the sensor element. The device further comprises means 408 for adjusting the gain of the electrical amplification circuits within the processor, in order to compensate for weak signals received from the sensor element.

Referring to FIGS. 12a to 12d, there is shown a diagrammatic representation of a sensor element assembly arranged with means for removing moisture from the sensor element to at least partially dry the sensor element between exhalations of the subject. Referring to FIG. 12a, a sensor element assembly is shown in plan view, with a cross-sectional view being shown in FIG. 12b. The sensor element assembly, generally indicated as 502 comprises a generally circular housing 504 having an inlet port 506 for oxygen or oxygenated air, a mouthpiece port 508 for connection to the mouth or nose of the subject and an outlet port 510 for exhaled gases. A one way valve 512 is disposed across the outlet port 510 and limits gas flow through the outlet port to the outwards direction, indicated by the arrow. A sensor element 514 is disposed centrally in the housing 504 and is connected to each port by radially extending channels within the housing, as shown.

In operation, a subject 516 breathes normally through the assembly 502, with the inhaled and exhaled gas streams passing out of and into the housing 504 through the mouthpiece port 508. Upon exhalation by the subject 516, the expired gas stream passes into the housing, contacts the sensor element 514 and leaves through the outlet port 510; the one way valve 512 opening to allow gases to escape from the housing. This flow pattern is shown schematically in FIG. 12c.

Upon inhalation by the subject 516, the one way valve 512 at the outlet port 510 closes, preventing the ingress of air into the housing through the outlet port. Oxygen or oxygen-enriched air is drawn into the housing through the inlet port 506 and flows along the radial channel to the sensor element 514 and then to the mouthpiece port 508 for the subject finally to inhale. The water content of the gas stream provided to the inlet port 506 is significantly lower than that of exhaled gases, typically being close to or at ambient conditions. Accordingly, the flow of the inhaled gas stream passing the sensor element 514 removes moisture from the sensor element, thus at least partially drying the sensor. This flow pattern is shown schematically in FIG. 12d.

Upon exhalation by the subject, the sensor element 514 is again contacted with an expired gas stream with a high moisture content, thus registering a further breath by the subject.

An alternative sensor element assembly is shown in FIGS. 13a and 13b. The sensor element assembly, generally indicated as 602, comprises a sensor element 604 mounted to a body 606. The body 606 is formed with various ports and channels therein, as follows. An inlet port 608 is provided for the supply of gas streams to be inhaled by the subject and extends laterally through the body 606. The inlet port 608 terminates in a first major port 610 extending through the body perpendicular to the inlet port 608 and away from the sensor element 604 for the supply of a gas stream to a subject. A second, minor port 612 extends towards the sensor element 604 and directs a jet of gas onto the surface of the sensor element bearing the electrodes and coating. A third port 614 extends through the body 606 substantially parallel to the first and second ports between the sensor element 604 and a subject.

The sensor element 604 is shown sealed to the opposing surface of the body 606 by a gas-tight seal 616 and arranged such that a channel for the flow of gas past the face of the sensor element between the sensor element and the body is formed. A one-way valve 618 is provided to control the direction of gas flowing past the sensor element.

In operation, a supply of gas, such as oxygen or oxygen-enriched air, is supplied to the inlet port 608. Upon inhalation by the subject, as shown in FIG. 13a, the gas stream flows along through the inlet port 608 and divides into two streams. A major portion of the supplied gas stream flows along the first port 610 and to the mouth or nose of the subject. However, a minor portion of the supplied gas stream passes along the third port 612 and forms a jet of gas impinging on and flowing past the surface of the sensor element 604. The action of this jet of gas is to remove moisture from the sensor element. The gas stream passing the surface of the sensor element is inhaled by the subject through the third port 614. During inhalation, the one-way valve 618 is closed, preventing the ingress of air into the body from other than the inlet port 608.

Upon exhalation by the subject, the gas stream exhaled by the subject passes along the third port 614 and impinges directly on the surface of the sensor element 604. The increase in pressure during exhalation by the subject is sufficient to open the one-way valve 618, allowing the expired gas stream to exit the assembly through the valve, as shown in FIG. 13b.

Referring to FIG. 14, there is shown a front view of a sensor element assembly of a further embodiment of the present invention. The assembly, generally indicated as 702 is shown in place on the face of a subject 704. The assembly 702 is shown in larger scale in side view in FIG. 15. As can be seen, the assembly 702 is held on the upper lip of the subject, between the mouth and the nose.

The assembly 702 comprises an elongate flexible support member 706 retained by a strap 708 of conventional design extending around the head of the subject. The support member extends evenly from the upper lip of the subject laterally to each side of the face of the subject. A gas conduit 710 is provided centrally below the nose and above the mouth of the subject. The conduit 710 is formed from an open ended tube having a generally semi-circular cross-section. In particular, the conduit 710 is opened at each end, that is adjacent the nose of the subject and above the mouth of the subject. A sensor element 712 is disposed and retained within the conduit, so as to have its surface exposed to a stream of gas flowing through the conduit.

An electrical cable 714 extends from the sensor element 712, along the strap 708 and over the left ear of the subject, as shown in FIG. 14. The cable 714 may be connected to a suitable host, control or display device. By routing the cable as shown in FIG. 14, the movement of the subject's head is not impeded.

In operation, the subject breathes normally through either the nose and/or mouth. It has been found that the conduit and sensor element arrangement as shown in FIGS. 14 and 15 captures a portion of the inhaled and exhaled gas stream, regardless of whether the subject is breathing nasally or orally. The captured portion is sufficient to allow the sensor element to react to changes in the moisture content of the gas stream and indicate the inhalations and exhalations, as discussed above.

### Example

A sensor assembly having the general configuration shown in FIGS. 7 and 8 was prepared. The electrodes were coated with an ion exchange layer comprising a commercially available sulphonated tetrafluoroethylene copolymer (Nafion®, ex Du Pont) and zeolite 4A. The coating was prepared as follows:

A suspension of the zeolite material was suspended in 10 ml of methanol. The zeolite had a uniform range of particle sizes, about 1 micron particle diameter.

The suspension was sonicated for 10 minutes, to ensure even dispersion of the Zeolite within the solution. An ultrasonic bath or probe may also be used. The electrode to be coated was then immersed into the solution and held for 2 seconds before withdrawal. The electrode was laid flat and the solvent allowed to naturally evapourate. Forced air convection may also be used to accelerate the evaporation of the solvent, if necessary.

The electrodes were inspected using scanning electron microscopy (SEM) to determine the distribution of zeolite particles across the electrodes. The results are shown in FIG. 16. As can be seen, the zeolite particles are finely dispersed across the surface of the electrode, with the spacing between particles generally being at least one particle diameter.

With the sensor still in the horizontal position, a minute volume of Nafion polymer was then dispensed onto the surface of the sensor using a syringe, and spread across the entire surface of the sensor using the edge of the syringe needle used to dispense the fluid. The solvent was again left to naturally evaporate away. The volume was such to ensure complete coverage of the surface area of the sensor, and to ensure that the resultant thickness of the film was as small as possible. Typical volumes range from 1 to 10 ul to cover an area of 1 cm², preferably 2 ul. The resultant thickness of the residual layer (after evaporation of the solvent) should be reasonably thin, consistent with the intended application. Practically, layer thicknesses of 10 to 1000 nm can be achieved using this method, preferably 100 nm.

The sensor element as described hereinbefore was exposed to a plurality of successive gas streams exhaled by a human subject. An indication of the concentration of water vapour was obtained as an output signal of the sensor element and displayed as a trace plotted against time. The display is shown in FIG. 17.

It can be seen that the patterns of water vapour concentration for successive breaths allow the respiration rate to be accurately determined and monitored.

1. A sensor assembly for monitoring the respiration pattern of a subject, the sensor comprising:
a sensor element for exposing to a gas stream exhaled by the subject comprising a first electrode, a second electrode and an active layer extending between the first and second electrodes to provide an electrical path therebetween, the sensor element being responsive to the concentration of water vapour in the gas stream exhaled by the subject, whereby the conductivity of the path between the electrodes varies in response to changes in the concentration of water vapour;
a processor for determining the pattern of respiration of the subject; and
means for displaying information relating to the determined respiration pattern to a user.

2. The sensor assembly according to claim 1, wherein the respiration pattern comprises the respiration rate of the subject, the processor determining the respiration rate of the subject.

3. The sensor assembly according to claim 1, wherein the respiration pattern comprises the depth of respiration of the subject, the processor determining the depth of respiration of the subject.

4. The sensor assembly according to claim 3, wherein the processor is adapted to integrate data generated by the sensor element relating to the concentration of water vapour and time of the exhalation to determine the depth of respiration.

5. The sensor assembly according to claim 1, wherein the sensor element is disposed in an assembly for attaching on or around the face of the subject.

6. The sensor assembly according to claim 5, wherein the sensor element is disposed within a mask to be worn by the subject.

7. The sensor assembly according to claim 5, wherein the sensor element is mountable on the upper lip of the subject.

8. The sensor assembly according to claim 7, wherein the sensor element is disposed within an open ended conduit for extending longitudinally between the mouth and nose of the subject.

9. The sensor assembly according to claim 5, wherein the sensor element is disposed in a line for supplying gases to the subject.

10. The sensor assembly according to claim 1, wherein the sensor element is adapted for location within the nasal passages or cannulae of the subject.

11. The sensor assembly according to claim 1, wherein the sensor element is wirelessly connected to the processor.

12. The sensor assembly according to claim 1, further comprising means to remove moisture from the sensor element between exhalations of the subject.

13. The sensor assembly according to claim 12, wherein the sensor element is disposed to be contacted by the gas stream being inhaled by the subject.

14. The sensor assembly according to claim 12, wherein the sensor element is disposed to be contacted by a stream of pressurized gas supplied to the sensor assembly.

15. The sensor assembly according to claim 1, wherein the active layer of the sensor element comprises a porous material.

16. The sensor assembly according to claim 15, wherein the porous material is a zeolite.

17. The sensor assembly according to claim 1, wherein the active layer comprises an ion-exchange material.

18. The sensor assembly according to claim 17, wherein the ion exchange material is a sulphonated tetrafluoroethylene copolymer.

19. The sensor assembly according to claim 17, wherein the active layer comprises an ion-exchange material and a zeolite, the zeolite being in the form of particles distributed in a fine dispersion through the ion exchange material.

20. The sensor assembly according to claim 1, wherein the active layer has a thickness of from 1 to 1000 nm, preferably from 5 to 500 nm, more preferably from 10 to 100 nm.

21. The sensor assembly according to claim 1, wherein the sensor element comprises a third electrode.

22. The sensor assembly according to claim 1, wherein the electrodes are of copper, gold or platinum.

23. The sensor assembly according to claim 1, wherein the electrodes are supported on an inert substrate.

24. The sensor assembly according to claim 23, wherein the electrodes are formed on the surface of the inert substrate by thick film screen printing.

25. The sensor assembly according to claim 1, wherein the electrodes each comprise a plurality of electrode portions, the electrode portions being arranged in an interdigitated array.

26. The sensor assembly according to claim 1, further comprising a sensor element responsive to one or more other components in the exhaled gas stream of the subject.

27. The sensor assembly according to claim 1, further comprising means for measuring the cardiac output of the subject.

28. The sensor assembly according to claim 27, wherein the said means are located in contact with the nasal septum of the subject.

29. The sensor assembly according to claim 28, further comprising means for stimulating the subject to change the level of arousal of the subject.

30. The sensor assembly according to claim 1, further comprising a CPAP device.

31. A method of monitoring the respiration pattern of a subject, the method comprising:
contacting a gas stream exhaled by the subject with the sensor element of an electrochemical sensor assembly, thereby generating an signal from the sensor element in response to changes in the concentration of water vapour in the exhaled gas stream;
processing the electrical signal to determine the respiration pattern of the subject; and
generating a display of information relating to the respiration pattern for a user.

32. The method according to claim 31, wherein the method comprises contacting the sensor element with exhaled gas streams during tidal breathing.

33. The method according to claim 31, wherein a voltage is applied across the electrodes of the electrochemical sensor assembly.

34. The method according to claim 33, wherein the voltage is a constant voltage.

35. The method according to claim 33, wherein the voltage is varied with time.

36. The method according to claim 31, wherein the method determines the respiration rate of the subject.

37. The method according to claim 31, wherein the method determines the depth of respiration of the subject.

38. The method according to claim 37, wherein the depth of respiration is determined by integration of the data relating to concentration of water vapour over time of the exhaled gas stream.

39. (canceled)

40. The method according to claim 31, further comprising stimulating the subject upon detection of an erratic respiration pattern.

41. The method according to claim 40, wherein the subject is stimulated sufficient to arouse them from REM sleep.

42. The method according to claim 31, further comprising administering a stream of pressurized gas to the nose and/or mouth of the subject upon detection of an erratic respiration pattern.

43. The method according to claim 31, wherein the respiration of the subject is monitored over a plurality of breaths.

44. The method according to claim 43, wherein the respiration of the subject is monitored during tidal breathing.

45. The method according to claim 31, wherein the method is used in diagnosing a subject with sleep apnea.

46. A system for the treatment of a subject with sleep apnea, the system comprising:
means for monitoring the respiration pattern of the subject;
means for administering to the subject a stream of pressurized gas sufficient to open the airways of the subject when activated by the means for monitoring the respiration pattern.

47. (canceled)

48. The system according to claim 46, further comprising means for stimulating the subject sufficient to change the level of arousal of the subject.

49. A method of treating a subject with sleep apnea, the method comprising the steps of:
monitoring the respiration pattern of the subject; and
upon detection of a erratic respiration pattern indicating the onset of an apneic event, initiating the supply of a stream of pressurized gas to the nose and/or mouth of the subject sufficient to maintain the airways of the subject open.

50. (canceled)

51. (canceled)

52. (canceled)

53. A system for providing a continuous positive airway pressure to a subject, the system comprising:
means for monitoring the respiration pattern of the subject;
means for delivering a stream of gas to the subject sufficient to maintain a continuous positive airway pressure within the subject; and
means for controlling the means for delivering the stream of gas responsive to the output from the means for monitoring the respiration pattern of the subject.

54. (canceled)

55. The method according to claim 31, wherein the method is used in training the metabolism of a subject.

56. The method according to claim 31, wherein the method is used in training the lung function of a subject.
